(19) United States
(12) Patent Application Publication
(10) Pub. No.: US 2025/0251409 A1
(43) Pub. Date: Aug. 7, 2025

(54) Automated Medical Diagnostic System and Method

(71) Applicant: Interdiagnostic Inc., Short Hills, NJ (US)

(72) Inventors: Michael Tu, Plano, TX (US); Prasanth Bijjam, Schaumburg, IL (US); Alexander Zasky, Mountain View, CA (US); Mark Fisher, Mountain View, CA (US); Eric Shain, Glencoe, IL (US); Craig Sampson, Mountain View, CA (US); Claire Zhou, Chicago, IL (US)

(21) Appl. No.: 18/918,967

(22) Filed: Oct. 17, 2024

Related U.S. Application Data
(63) Continuation of application No. 17/332,823, filed on May 27, 2021, now abandoned.
Application No. 17/332,823 is a continuation-in-part of application No. 16/712,748, filed on Dec. 12, 2019, now abandoned.
Application No. 16/712,748 is a continuation-in-part of application No. 16/015,417, filed on Jun. 22, 2018, now Pat. No. 11,397,176.
(60) Provisional application No. 62/524,199, filed on Jun. 23, 2017.
(60) Provisional application No. 62/779,560, filed on Dec. 14, 2018.
(60) Provisional application No. 62/802,768, filed on Feb. 8, 2019.
(60) Provisional application No. 62/823,939, filed on Mar. 26, 2019.
(60) Provisional application No. 62/848,107, filed on May 15, 2019.
(60) Provisional application No. 62/866,067, filed on Jun. 25, 2019.
(60) Provisional application No. 62/937,852, filed on Nov. 20, 2019.
(60) Provisional application No. 63/031,011, filed on May 28, 2020.
(60) Provisional application No. 63/044,630, filed on Jun. 26, 2020.
(60) Provisional application No. 63/092,819, filed on Oct. 16, 2020.
(60) Provisional application No. 63/116,201, filed on Nov. 20, 2020.
(60) Provisional application No. 63/181,043, filed on Apr. 28, 2021.

(51) Int. Cl. G01N 35/00; G01N 35/04; G01N 35/10; G16H 10/40; G16H 40/63

(52) U.S. Cl. CPC G01N 35/00029; G01N 35/00732; G01N 35/00871; G01N 35/0099; G01N 35/04; G01N 35/1009; G16H 10/40; G16H 40/63; G01N 2035/00108; G01N 2035/00168; G01N 2035/00306; G01N 2035/00752; G01N 2035/00881; G01N 2035/0403

(57) ABSTRACT

The present disclosure relates to automated medical diagnostic systems and methods. One example embodiment includes a system. The system includes a test cartridge, a test strip usable to indicate the presence of one or more patient conditions, and a kiosk configured to receive and process the test cartridge and the test strip. The kiosk includes a vortex mixer; a conveyor belt; a robotic pipette module; an imaging system; a display; and a processor communicative coupled to the vortex mixer, the conveyor belt, the robotic pipette module, the imaging system, and the display. The processor is configured to execute instructions stored within a memory to operate the vortex mixer, the conveyor belt, the robotic pipette module, the imagine system, and the display; to receive the image of the test strip from the imaging system; and to analyze the image to determine whether one or more patient conditions is present.

### CROSS-REFERENCE TO RELATED APPLICATIONS

The present application is a continuation application claiming priority to U.S. Non-Provisional patent application Ser. No. 17/332,823, filed May 27, 2021; which is a continuation-in-part application claiming priority to U.S. Non-Provisional patent application Ser. No. 16/712,748, filed Dec. 12, 2019; which is a continuation-in-part application claiming priority to U.S. Non-Provisional patent application Ser. No. 16/015,417, filed Jun. 22, 2018 and issued as U.S. Pat. No. 11,397,176 on Jul. 26, 2022; which itself claims the benefit of U.S. Provisional Patent Application No. 62/524,199, filed Jun. 23, 2017. U.S. Non-Provisional patent application Ser. No. 16/712,748 also claims the benefit of U.S. Provisional Patent Application No. 62/779,560, filed Dec. 14, 2018; U.S. Provisional Patent Application No. 62/802,768, filed Feb. 8, 2019; U.S. Provisional Patent Application No. 62/823,939, filed Mar. 26, 2019; U.S. Provisional Patent Application No. 62/848,107, filed May 15, 2019; U.S. Provisional Patent Application No. 62/866,067, filed Jun. 25, 2019; and U.S. Provisional Patent Application No. 62/937,852, filed Nov. 20, 2019. Additionally, U.S. Non-Provisional patent application Ser. No. 17/332,823 claims the benefit of U.S. Provisional Patent Application No. 63/031,011, filed May 28, 2020; U.S. Provisional Patent Application No. 63/044,630, filed Jun. 26, 2020; U.S. Provisional Patent Application No. 63/092,819, filed Oct. 16, 2020; U.S. Provisional Patent Application No. 63/116,201, filed Nov. 20, 2020; and U.S. Provisional Patent Application No. 63/181,043, filed Apr. 28, 2021. The contents of U.S. patents application Ser. Nos. 17/332,823, 16/712,748, 16/015,417, 62/524, 199, 62/779,560, 62/802,768, 62/823,939, 62/848, 107, 62/866,067, 62/937,852, 63/031,011, 63/044,630, 63/092,819, 63/116,201, and 63/181,043 and U.S. Pat. No. 11,397,176 are hereby incorporated by reference.

### TECHNICAL FIELD

The present disclosure relates to a medical diagnostic system that automates analysis of samples to predict a medical condition.

### BACKGROUND

Unless otherwise indicated herein, the materials described in this section are not prior art to the claims in this application and are not admitted to be prior art by inclusion in this section.

Currently, there exist different systems and methods of performing rapid, point-of-care colorimetric-based or fluorescent-based lateral flow immunoassay strip tests and chemistry-based strip tests.

Existing systems and methods are inefficient in high-volume testing environments. Existing diagnostic platforms require operators to either actively manage multiple test strips and multiple timers when test strips are manually timed and read, or actively manage multiple analyzers, when test strips are processed and read by a point-of-care, serial processing analyzer one at a time. A parallel processing analyzer may be used instead. Multiple test strips could be processed in parallel to increase testing throughput and simplify operational complexity.

Existing systems and methods rely on a healthcare worker to be physically present to prepare patient samples and initiate testing. A patient-initiated, point-of-care platform that automates a range of rapid diagnostic tests across various biofluids may be used instead. A patient-initiated platform could expand the settings in which point-of-care tests can be performed to community-based or near-home locations, outside of the hospital or clinic.

Clinical diagnostics play an important role in the management of infectious disease outbreaks such as the COVID-19 pandemic. Widespread testing, alongside contract tracing and stringent social distancing measures, has been shown to limit the community spread of SARS-CoV-2. Point-of-care, lateral flow antigen immunoassay tests have become a priority due to their speed and low cost. However, as of now, SARS-CoV-2 antigen tests are being delivered through platforms that were originally designed for low-volume testing in traditional healthcare settings like hospitals and clinics. These include test platforms that require a serial processing analyzer and test platforms without instrumentation. In practice, these test platforms are inefficient and difficult to scale for high-volume testing in the community and for high-volume screening programs in workplaces and schools. Further, tests that are performed without instrumentation can be labor-intensive and introduce user errors.

There exist serial processing analyzers that automate the processing and analysis of a test strip serially, one at a time. These analyzers are compatible with companion systems that send data from the analyzer to a Laboratory Information System (LIS), Electronic Health Record (EHR) system, or another Healthcare Insurance Portability and Accountability Act (HIPAA)-compliant database on a cloud server. Serial processing analyzers can be set to WALK AWAY mode, which enables the operator to place the test strip into the analyzer for processing immediately after sample application and analysis after pre-determined amount of processing time has passed, or READ NOW mode, which enables the operator the place the test strip into the analyzer once the sample has been applied, and the test strip has processed a pre-determined amount of time. In practice, neither of these modes are suited to high-volume testing. In WALK AWAY mode, analyzers have an occupancy time equal to the processing time of the test strip after sample application. Since each analyzer is only able to process one test strip at a time, multiple analyzers would be required to perform higher volumes of testing. In READ NOW mode, operators can increase throughput by manually timing test strip processing outside of the analyzer with separate timers before inserting test strips into the analyzer for analysis. However, manual timing can introduce operational complexity and user errors from the simultaneous management of multiple test strips and timers. It can also increase personnel costs due to the active management of a manual process. In both modes, serial processing analyzers also require operators to remove a previously analyzed test strip before inserting a new test strip for analysis.

There also exist techniques for testing test strips without instrumentation. Manual timing of test strips can introduce operational complexity and user errors from the simultaneous management of many test strips and timers. User errors from manual processes can also include inaccurate visual interpretation and incorrect entry of results into the EHR system. Government spot checks of facilities that conduct point-of-care tests generally have found less than 50% compliance with policies meant to ensure proper testing procedures. Further, tests that are performed without instrumentation are more labor-intensive as they require an operator to manually perform all steps in the testing process.

In recent years, healthcare has been transitioning to a more decentralized care delivery model, with services shifting away from the hospital and into the community. Telehealth and retail-based health services have emerged as accessible, cost-effective, and time-saving alternatives to basic hospital care. The benefits of these alternatives are compounded in rural areas, where residents may live far away from hospitals. From 2017 to 2018, telehealth and retail clinic use grew by 12% and 10%, respectively, while emergency department use fell by 15%. Telehealth providers do not have access to point-of-care testing, limiting the range of health issues that can be diagnosed and treated through at-home or near-home telehealth. Telehealth providers must instead send patients to a central laboratory for testing, which delays patient care.

Therefore, a need exists to solve the deficiencies present in the prior art. What is needed is a system and method to facilitate testing of samples for biomarkers indicative of a medical condition. What is needed is a system and method that automatically mixes sample within a sample container with reagent(s) within a sample container. What is needed is a system and method for automatically extracting a sample or sample mixed with reagent(s) from a sample container. What is needed is a system and method to automate biofluid mixing, preparation and handling within a sample container. What is needed is a system and method to automate fluid handling and transportation of a sample mixed with reagent(s) from the sample container to test strip(s). What is needed is a system and method that automatically controls the sample volume dispensed onto test strip(s). What is needed is a system and method to automatically facilitate the parallel processing of multiple colorimetric-based or fluorescent-based lateral flow immunoassay test strips or chemistry-based test strips for automated testing at the point-of-care. What is needed is a system and method to automate testing using indicators that can be detected through optical or fluorescent methods. What is needed is a system and method to communicate detected biomarkers indicative of a condition to a network-connected electronic computing device and/or network-connected database. What is needed is a system and method of collecting, processing, and analyzing biofluids that can be performed by a patient or caregiver. What is needed is a system and method that allows a patient or caregiver to place test strips and sample containers in or on an automated analyzer. What is needed is a system and method of automating the processing of tests, optically analyzing indicators to predict a medical condition and automating the disposal of analyzed test strips. What is needed is a system and method of automating the disposal of processed sample containers. What is needed is a system and method of automatically detecting indicators for SARS-CoV-2, Influenza A+B, and other infectious diseases or medical conditions within an acceptable margin of error. What is needed is a system and method for a telehealth or non-telehealth clinicians to remotely pre-authorize and initiate a programmatic healthcare intervention based on a test result, including, but not limited to, placing a prescription order.

### SUMMARY

The specification and drawings disclose embodiments that relate to a medical diagnostic system that automates analysis of samples to predict a medical condition. Embodiments disclosed herein will allow patients, caregivers, trained healthcare professionals and/or trained non-healthcare professionals to directly perform point-of-care lateral flow immunoassay strip-based tests and/or chemistry-based strip-based tests for clinical diagnosis by a healthcare provider and/or enable high-throughput processing of rapid point-of-care lateral flow immunoassay strip-based tests and/or chemistry-based strip-based tests.

In a first aspect, the disclosure describes a system. The system includes a test cartridge. The test cartridge includes a first chamber configured to store a patient sample and a buffer extraction solution. The test cartridge also includes a second chamber configured to store a pipette tip. The system also includes a test strip usable to indicate the presence of one or more patient conditions. Additionally, the system includes a kiosk configured to receive and process the test cartridge and the test strip. The kiosk includes a vortex mixer configured to mix the patient sample with the buffer extraction solution to generate a mixture. The kiosk also includes a conveyor belt. The conveyor belt is configured to receive the test strip at a first location within the kiosk. The conveyor belt is also configured to transfer the test strip from the first location to a second location within the kiosk. Additionally, the conveyor belt is configured to transfer the test strip from the second location to a third location within the kiosk. In addition, the kiosk includes a robotic pipette module configured to retrieve the pipette tip from the second chamber and dispense at least a portion of the mixture onto the test strip using the pipette tip while the test strip is located at the second location. Further, the kiosk includes an imaging system configured to capture an image of the test strip while the test strip is located at the third location. Yet further, the kiosk includes a display configured to display instructions regarding using the test cartridge, the test strip, or the kiosk. Even further, the kiosk includes a processor communicatively coupled to the vortex mixer, the conveyor belt, the robotic pipette module, the imaging system, and the display. The processor is configured to execute instructions stored within a memory to: operate the vortex mixer; operate the conveyor belt; operate the robotic pipette module; operate the imaging system; receive the image of the test strip from the imaging system; analyze the image of the test strip to determine whether at least one of the one or more patient conditions is present; and operate the display.

In a second aspect, the disclosure describes a kiosk configured to receive and process a test cartridge and a test strip. The kiosk includes a vortex mixer configured to mix a patient sample with a buffer extraction solution to generate a mixture. The test cartridge includes a first chamber configured to store the patient sample and the buffer extraction solution. The test cartridge also includes a second chamber configured to store a pipette tip. The kiosk also includes a conveyor belt. The conveyor belt is configured to receive the test strip at a first location within the kiosk. The test strip is usable to indicate the presence of one or more patient conditions. The conveyor belt is also configured to transfer the test strip from the first location to a second location within the kiosk. In addition, the conveyor belt is configured to transfer the test strip from the second location to a third location within the kiosk. Additionally, the kiosk includes a robotic pipette module configured to retrieve the pipette tip from the second chamber and dispense at least a portion of the mixture onto the test strip using the pipette tip while the test strip is located at the second location. Further, the kiosk includes an imaging system configured to capture an image of the test strip while the test strip is located at the third location. In addition, the kiosk includes a display configured to display instructions regarding using the test cartridge, the test strip, or the kiosk. Still further, the kiosk includes a processor communicatively coupled to the vortex mixer, the conveyor belt, the robotic pipette module, the imaging system, and the display. The processor is configured to execute instructions stored within a memory to: operate the vortex mixer; operate the conveyor belt; operate the robotic pipette module; operate the imaging system; receive the image of the test strip from the imaging system; analyze the image of the test strip to determine whether at least one of the one or more patient conditions is present; and operate the display.

In a third aspect, the disclosure describes a test cartridge. The test cartridge includes a first chamber configured to store a patient sample and a buffer extraction solution. The test cartridge also includes a second chamber configured to store a pipette tip. The test cartridge is configured to be received and processed by a kiosk along with a test strip usable to indicate the presence of one or more patient conditions. The kiosk includes a vortex mixer configured to mix the patient sample with the buffer extraction solution to generate a mixture. The kiosk also includes a conveyor belt. The conveyor belt is configured to receive the test strip at a first location within the kiosk. The conveyor belt is also configured to receive the test strip at a first location within the kiosk. Additionally, the conveyor belt is configured to transfer the test strip from the second location to a third location within the kiosk. Further, the kiosk includes a robotic pipette module configured to retrieve the pipette tip from the second chamber and dispense at least a portion of the mixture onto the test strip using the pipette tip while the test strip is located at the second location. In addition, the kiosk includes an imaging system configured to capture an image of the test strip while the test strip is located at the third location. Still further, the kiosk includes a display configured to display instructions regarding using the test cartridge, the test strip, or the kiosk. Additionally, the kiosk includes a processor communicatively coupled to the vortex mixer, the conveyor belt, the robotic pipette module, the imaging system, and the display. The processor is configured to execute instructions stored within a memory to: operate the vortex mixer; operate the conveyor belt; operate the robotic pipette module; operate the imaging system; receive the image of the test strip from the imaging system; analyze the image of the test strip to determine whether at least one of the one or more patient conditions is present; and operate the display.

In a fourth aspect, the disclosure describes a method. The method includes receiving, by a kiosk, a test cartridge. The test cartridge includes a first chamber configured to store a patient sample and a buffer extraction solution. The test cartridge also includes a second chamber configured to store a pipette tip. The method also includes receiving, by the kiosk, a test strip usable to indicate the presence of one or more patient conditions. Additionally, the method includes displaying, by a display of the kiosk, instructions regarding using the test cartridge, the test strip, or the kiosk. Further, the method includes processing, by the kiosk, the test cartridge and the test strip. Processing the test cartridge and test strip includes mixing, by a vortex mixer, the patient sample with the buffer extraction solution to generate a mixture. Processing the test cartridge and test strip also includes receiving, by a conveyor belt, the test strip at a first location within the kiosk. Additionally, processing the test cartridge and test strip includes transferring, by the conveyor belt, the test strip from the first location to a second location within the kiosk. Further, processing the test cartridge and test strip includes retrieving, by a robotic pipette module, the pipette tip from the second chamber. In addition, processing the test cartridge and test strip includes dispensing, by the robotic pipette module, at least a portion of the mixture onto the test strip using the pipette tip while the test strip is located at the second location. Still further, processing the test cartridge and test strip includes transferring, by the conveyor belt, the test strip from the second location to a third location within the kiosk. Even further, processing the test cartridge and test strip includes capturing, using an imaging system, an image of the test strip while the test strip is located at the third location. Yet further, processing the test cartridge and test strip includes receiving, by a processor executing instructions stored within a memory, the image of the test strip from the imaging system. Still yet further, processing the test cartridge and test strip includes analyzing, by the processor executing the instructions stored within a memory, the image of the test strip to determine whether at least one of the one or more patient conditions is present.

Terms and expressions used throughout this disclosure are to be interpreted broadly. Terms are intended to be understood respective to the definitions provided by this specification. Technical dictionaries and common meanings understood within the applicable art are intended to supplement these definitions. In instances where no suitable definition can be determined from the specification or technical dictionaries, such terms should be understood according to their plain and common meaning. However, any definitions provided by the specification will govern above all other sources.

Various objects, features, aspects, and advantages described by this disclosure will become more apparent from the following detailed description, along with the accompanying drawings in which like numerals represent like components.

The foregoing summary is illustrative only and is not intended to be in any way limiting. In addition to the illustrative aspects, embodiments, and features described above, further aspects, embodiments, and features will become apparent by reference to the figures and the following detailed description.

### BRIEF DESCRIPTION OF THE FIGURES

FIG. 1A is a high-throughput reader, according to example embodiments.

FIG. 1B is a high-throughput reader, according to example embodiments.

FIG. 1C is a high-throughput reader, according to example embodiments.

FIG. 2 is a self-service diagnostic kiosk and self-testing kit, according to example embodiments.

FIG. 3 is a patient/user workflow for a self-service diagnostic kiosk and self-testing kit, according to example embodiments.

FIG. 4 is a sample analysis process for a self-service diagnostic kiosk, according to example embodiments.

FIG. 5A is a test cartridge, according to example embodiments.

FIG. 5B is a test cartridge, according to example embodiments.

FIG. 6 is a self-testing kit, according to example embodiments.

FIG. 7 is a self-service diagnostic kiosk, according to example embodiments.

FIG. 8 is a self-service diagnostic kiosk, according to example embodiments.

FIG. 9 is a test cartridge insertion port on a self-service diagnostic kiosk, according to example embodiments.

FIG. 10 is a test cartridge, according to example embodiments.

FIG. 11 is a self-service diagnostic kiosk, according to example embodiments.

FIG. 12 is a self-service diagnostic kiosk, according to example embodiments.

FIG. 13A is a self-service diagnostic kiosk before a test cartridge is loaded into it, according to example embodiments.

FIG. 13B is a self-service diagnostic kiosk after a test cartridge is loaded into it, according to example embodiments.

FIG. 13C is a self-service diagnostic kiosk while vortex mixer is mixing the sample and buffer extraction solution in the test cartridge together, according to example embodiments.

FIG. 13D is a self-service diagnostic kiosk while the robotic pipette module is picking up the pipette tip from the test cartridge, according to example embodiments.

FIG. 13E is a self-service diagnostic kiosk while the robotic pipette module is using the pipette tip to aspirate a fixed volume of mixed sample from the test cartridge, according to example embodiments.

FIG. 13F is a self-service diagnostic kiosk while the robotic pipette module is using the pipette tip to dispense a fixed volume of mixed sample onto the test strip, according to example embodiments.

FIG. 13G is a self-service diagnostic kiosk after the robotic pipette module has returned the used pipette tip to its original location in the test cartridge, according to example embodiments.

FIG. 13H is a self-service diagnostic kiosk while the test result is developing on the test strip and the conveyor belt is advancing the test strip towards the imaging module, according to example embodiments.

FIG. 13I is a self-service diagnostic kiosk while the test strip is being imaged by the imaging module, according to example embodiments.

FIG. 14A is a self-service diagnostic kiosk, according to example embodiments.

FIG. 14B is a self-service diagnostic kiosk, according to example embodiments.

FIG. 15A is a step of the workflow for a self-service diagnostic kiosk, according to example embodiments.

FIG. 15B is a step of the workflow for a self-service diagnostic kiosk, according to example embodiments.

FIG. 15C is a step of the workflow for a self-service diagnostic kiosk, according to example embodiments.

FIG. 15D is a step of the workflow for a self-service diagnostic kiosk, according to example embodiments.

FIG. 15E is a step of the workflow for a self-service diagnostic kiosk, according to example embodiments.

FIG. 16 is a deterministic geometric image processing algorithm for processing and analyzing images of colorimetric, qualitative test strip results: Influenza A+B test, according to example embodiments.

FIG. 17A is a deterministic geometric image processing algorithm for processing and analyzing images of colorimetric, qualitative test strip results: SARS-CoV-2 test, according to example embodiments.

FIG. 17B is a deterministic geometric image processing algorithm for processing and analyzing images of colorimetric, qualitative test strip results: SARS-CoV-2 test, according to example embodiments.

FIG. 18 is a computing device, according to example embodiments.

FIG. 19A is a flow chart illustration of a method, according to example embodiments.

FIG. 19B is a flow chart illustration of a method, according to example embodiments.

### DETAILED DESCRIPTION

Example methods and systems are described herein. Any example embodiment or feature described herein is not necessarily to be construed as preferred or advantageous over other embodiments or features. The example embodiments described herein are not meant to be limiting. It will be readily understood that certain aspects of the disclosed systems and methods can be arranged and combined in a wide variety of different configurations, all of which are contemplated herein.

Furthermore, the particular arrangements shown in the Figures should not be viewed as limiting. It should be understood that other embodiments might include more or less of each element shown in a given Figure. In addition, some of the illustrated elements may be combined or omitted. Similarly, an example embodiment may include elements that are not illustrated in the figures.

The following disclosure is provided to describe various embodiments of a medical diagnostic system. Skilled artisans will appreciate additional embodiments and uses of the present invention that extend beyond the examples of this disclosure. Terms included by any claim are to be interpreted as defined within this disclosure. Singular forms should be read to contemplate and disclose plural alternatives. Similarly, plural forms should be read to contemplate and disclose singular alternatives. Conjunctions should be read as inclusive except where stated otherwise.

Expressions such as “at least one of A, B, and C” should be read to permit any of A, B, or C singularly or in combination with the remaining elements. Additionally, such groups may include multiple instances of one or more element in that group, which may be included with other elements of the group. All numbers, measurements, and values are given as approximations unless expressly stated otherwise.

### I. Example Embodiments

Various aspects of the present disclosure will now be described in detail, without limitation. In the following disclosure, a medical diagnostic system that automates analysis of samples to predict a medical condition will be discussed. Those of skill in the art will appreciate alternative labeling of the medical diagnostic system as a self-service diagnostic kiosk, kiosk, diagnostic kiosk, platform, a high-throughput reader, a self-service platform, self-service diagnostic platform,, or other similar names. Skilled readers should not view the inclusion of any alternative labels as limiting in any way.

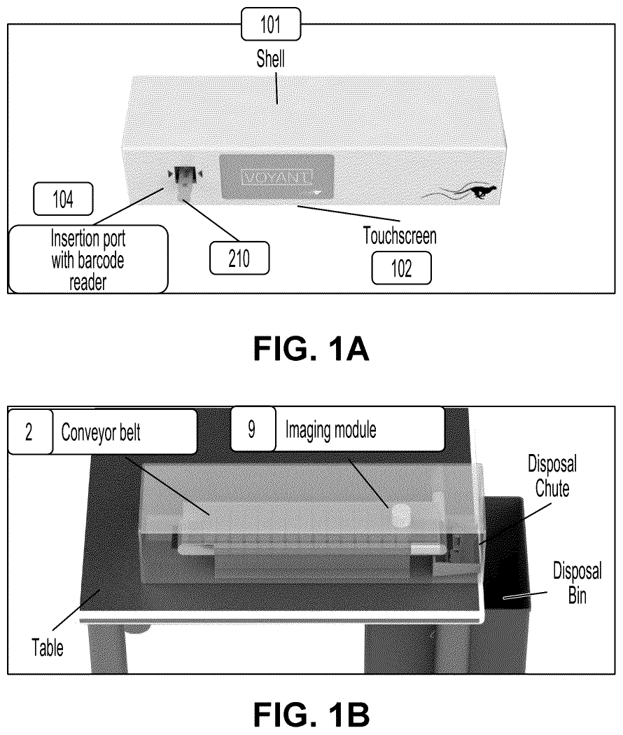

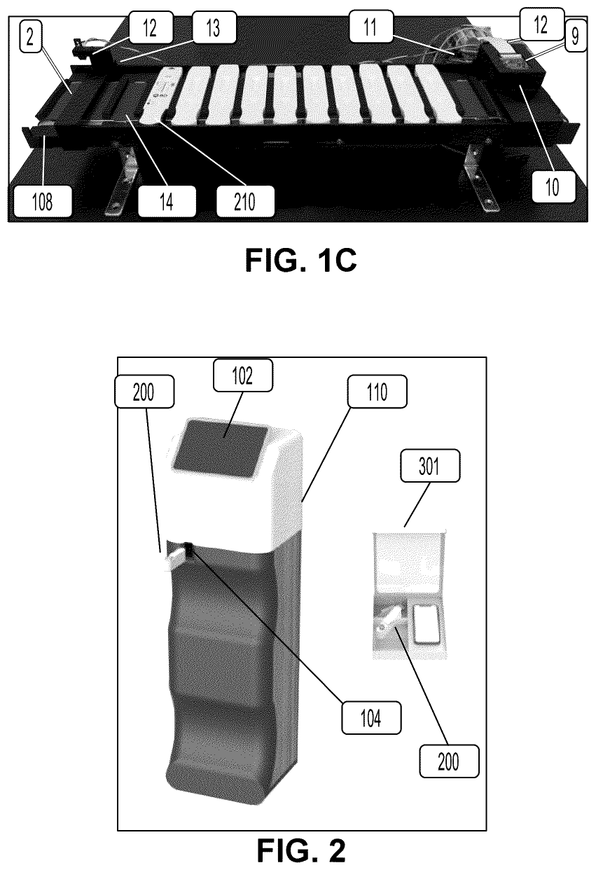

Referring now to FIG. 1 an embodiment of a high-throughput reader will now be discussed. In this embodiment, a sample would be manually prepared and manually applied to the test strip prior to insertion of the test strip into the device.

A method of connecting a unique operator or user ID to each shift, or set of test strips, will be described. A camera on the device or a smartphone camera may be activated through a mobile application to image the unique operator or user ID in the form of a barcode or Quick Response (QR) code. Existing libraries such as, but not limited to, zbar and zxing may be used to process barcodes or QR codes.

Another method of connecting a unique operator or user ID to each shift, or batch of tests, will be described. The unique operator or user ID may be scanned by a companion scanner or companion camera. Existing libraries such as but not limited to zbar and zxing may be used to process barcodes or QR codes.

A method of connecting a unique operator or user ID, a unique patient ID and a unique test ID will be discussed. The unique patient ID will be printed by the healthcare provider. A camera on the device or a smartphone camera may be activated through a mobile application to first image the unique patient ID and then image a unique barcode or QR code on the test strip. The detected visual codes will be interpreted by algorithms to record the unique patient ID and determine the test type and unique test ID from the unique barcode or QR code on the test strip. Existing libraries such as but not limited to zbar and zxing may be used to process barcodes or QR codes. The previously recorded unique operator or user ID, unique patient ID, test type, and unique test ID will be sent to a LIS, EHR system, or another HIPAA-compliant database on a cloud server database.

An alternative method of connecting a unique operator or user ID, unique patient ID, and a unique test ID will be discussed. The unique patient ID and unique barcode or QR code on the test strip may be scanned by a companion scanner or companion camera. Existing libraries such as but not limited to zbar and zxing may be used to process barcodes or QR codes. The previously recorded unique operator or user ID, unique patient ID, test type, and unique test ID will be sent to a LIS, EHR system, or another HIPAA-compliant database on a cloud server database.

An alternative method of connecting a unique operator or user ID, a unique patient ID, and a unique test ID will be discussed. The unique operator or user ID, unique patient ID, test type, and unique test ID may be manually inputted on a digital keyboard on a touch-sensitive, liquid-crystal display (LCD), a light-emitting diode (LED) display, or a cathode ray tube (CRT) monitor. The previously recorded unique operator or user ID, unique patient ID, test type, and unique test ID will be sent to a LIS, EHR system, or another HIPAA-compliant database on a cloud server database.

Referring now to FIG. 1, a method of test strip insertion will now be discussed. The test strip 210 will be placed into a test strip insertion port 104 by the user and onto a conveyor belt mechanism 2. A test strip may be inserted periodically, such as, but not limited to, once every minute or once every 40 seconds, depending on the test strip. As an example, a SARS-CoV-2 lateral flow test strip may be inserted once a minute. As another example, an Influenza A+B lateral flow tests trip may be inserted once every 40 seconds. Other example test strips, sample containers, and/or swabs are also possible. For example, test strips, sample containers, or swabs that test for Group A streptococcal infection (Strep A) using saliva, human chorionic gonadotropin (hCG) using urine, chlamydia (e.g., via vaginal swab), gonorrhea (e.g., via vaginal swab), Helicobacter pylori, and Clostridium difficile could also be inserted, among others. When the test strip is inserted into the test strip port, the port may sense the test strip has been inserted through a load cell, a touch sensor, or an optical sensor. Optointerrupter(s) can be used to detect when a test strip has been inserted. The conveyor will move periodically, such as, but not limited to, once every 60 seconds or once every 40 seconds, depending on the test strip. The movement of the conveyor will advance an inserted test strip towards the device camera for analysis. The movement of the conveyor will also bring an empty test strip holding slot in front of the test strip insertion port to hold the next inserted test strip. The mechanical design of the port and test strip cartridge may be optimized to ensure there is no sample contamination from test strip to test strip.

An alternative method of test strip insertion will now be discussed. A test strip port may be configured to allow a test to be placed into the port immediately after the previous test strip. When a test strip has been inserted into the test strip insertion port, and the insertion is detected by the device by sensors (e.g., one or more optointerruptors 12), the conveyor will immediately move and advance forward one slot 14 (e.g., based on one or more signals from a microcontroller 11) unless there is a test strip on the conveyor occupying the slot right before the imaging system 9.

An alternative method of test strip insertion will now be discussed. A test strip port may be configured to allow multiple test strips to be placed at once. The test strip insertion port opening can span the width of multiple test strip holding slots to allow the user to insert multiple test strips at a time. As an example, the test strip insertion port opening can span the width of three test strip holding slots to allow the user to insert three test strips at a time.

Referring now to FIG. 1, a method of determining the test type will be discussed. A unique barcode or QR code on the test strip will be scanned to determine the test type will now be discussed. As the test strip is inserted into the device port, the unique barcode or QR code on the test strip will be scanned by a visual reader (e.g., an imaging system or a barcode scanner) on the device. The detected visual code will be interpreted by the device using some reading algorithm to determine the test type. Existing libraries such as but not limited to zbar and zxing may be used to process barcodes or QR codes. The test type will set the processing time and the algorithm used to read the test strip. The frequency with which the conveyor moves may be equal to the processing time of the test strip divided by the number of test strip holding slots on the conveyor. As an example, when the test type is determined to be a SARS-CoV-2 test strip, the processing time will be set to 15 minutes. If the device is configured with 15 test strip holding spots, the conveyor would move once a minute for SARS-CoV-2 test strips. The SARS-CoV-2 deterministic geometric image processing algorithm would be used to analyze the test strip when the test strip reaches the test strip analysis reader. As another example, when the test type is determined to be an Influenza A+B test strip, the processing time will be set to 10 minutes. If the device is configured with 15 test strip holding slots, the conveyor would move every 40 seconds for Influenza A+B test strips. The Influenza A+B deterministic geometric image processing algorithm would be used to analyze the test strip when the test strip reaches the test strip analysis reader.

Referring now to FIG. 1, a parallel processing, conveyor belt mechanism will now be discussed. The mechanism may include a conveyor belt 2, a motor 13 to control the conveyor belt, cleats that form test strip holder slots 14 on the conveyor belt, test strip insertion port 104, a test strip analysis sensor (e.g., imaging system) 9, such as an optical reader (camera) or fluorescent reader, an opening and/or a disposal chute to dispose of test strips into a waste bin, test strip positioning sensors such as optointerrupters 12, and/or a communicative coupling with a microprocessor of the kiosk 11. The conveyor belt 2 allows multiple test strips to be inserted into the device over time, parallel processed over time, and develop results over time simultaneously before being analyzed by the test strip analysis sensor. The device may be configured to perform different types of tests, and the conveyor belt mechanism may be reconfigurable based on the processing time of different test types. The motor 13, such as a stepper motor or a direct current (DC) motor, may be configured to move the conveyor belt forward and/or backward to ensure each test strip is positioned underneath the test strip analysis sensor after the amount of processing time appropriate for each test type. Further, the conveyor belt may be reconfigurable based on different test strip sizes and formats, including, but not limited to, plastic test cassettes, paper test cards, and unhoused test strips, by changing the spacing of cleats on the conveyor belt. The cleats form test strip holders across the conveyor belt. It is understood that, in some embodiments, a timing belt (or other belt) could equally be used in addition to or instead of a conveyor belt.

Referring now to FIG. 1, a mechanism for parallel processing test strips will be discussed. A horizontally positioned conveyor belt 2 and a motor 13 may be configured to carry multiple test strips between test strip holding slots and cleats and to move the test strips by rotation (e.g., from a first location in a kiosk, to a second location in a kiosk, to a third location in a kiosk, etc.). It is understood that the conveyor belt may also operate in reverse (e.g., to transfer test strips from the third location in the kiosk to the second location in the kiosk). The conveyor will move periodically and/or as new test strips are inserted to move inserted test strips towards the imaging system as the test strip results develop over time. Positioning of the test strips as the conveyor moves can be controlled using sensors such as optointerrupters 12 that detect the presence of test strips and/or cleats. Optointerrupters may include an emitter-receiver pair, where the emitter emits electromagnetic waves such as infrared waves. When an object is under the emitter, the electromagnetic waves may reflect off the surface of the object and be detected by the receiver. Optointerrupters may communicate this detection with the kiosk's microcontroller via a digital signal. The sensors, such as optointerrupters 12, can be placed at different locations, such as by the test strip insertion port 104 or by the test strip analysis imager 9. After a determined amount of time, the conveyor will bring the test strip underneath an imaging system 9 where the test strip is imaged.

A positioning algorithm for positioning a test strip 210 (FIG. 1) or test cartridge 200 (FIGS. 2, 5, 8) underneath an imaging module 9 will now be discussed. In order to center the test strip or test cartridge precisely under the camera of the imaging module, the positioning algorithm uses the conveyor belt 2 and the optical imaging module 9. By continuously reading the video feed from the optical imaging module, the algorithm indexes the test strip or test cartridge on the conveyor belt forward until the algorithm detects the test strip in the video feed. The test strip is detected when the pixel intensity across the width of an image is greater than the pixel intensity of the background. Once the test strip is detected, the algorithm indexes the conveyor belt until there is an equal number of background pixels on each side of the test strip in the image, which corresponds to the test strip being perfectly centered. If the test strip is indexed too far, the algorithm indexes the conveyor belt backwards until the test strip is centered.

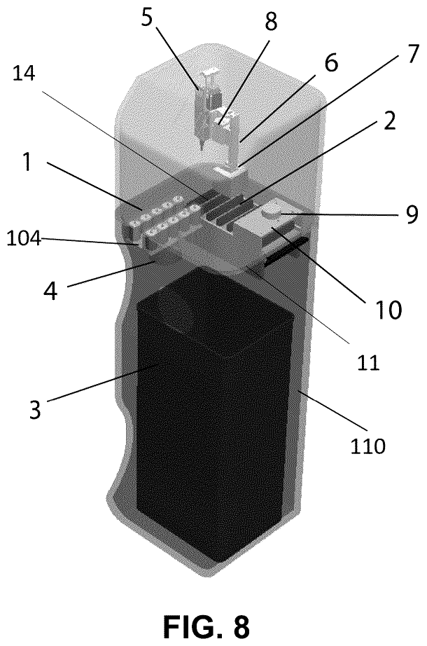

Referring now to FIG. 8, the conveyor belt 2 may move the test strip through an automated motion into a waste receptacle (e.g., an internal waste bin) 3 for disposal once an image of the test strip has been captured by the imaging system 9. In some embodiments, the test strip and/or an associated test cartridge may be converted from medical waste to municipal waste using a disinfectant prior to depositing the test strip and/or associated test cartridge into the kiosk waste receptacle.

An alternative method of parallel processing test strips using a horizontally positioned conveyor belt will be discussed. The test strips will be placed sideways onto the conveyor and test strip holder to improve space efficiency. The test strips can be placed sideways from any of the sides of the test strip.

An alternative method of parallel processing test strips using a vertically positioned conveyor belt will be discussed. This configuration allows large test strips, such as those in the format of large test cards, to be inserted into the conveyor space efficiently, and the configuration allows the large test strips to lay flat and horizontally while processing for optimal lateral fluid flow within the test strip.

An alternative method of parallel processing test strips using a diagonally positioned conveyor belt will be discussed. This configuration allows large test strips, such as those in the format of large test cards, to be inserted into the conveyor space efficiently, and the configuration allows the large test strips to lay flat and horizontally while processing for optimal lateral fluid flow within the test strip.

An alternative method of parallel processing test strips using a conveyor arranged as a carousel will be discussed. This configuration allows a higher number of test strips to be inserted into the conveyor, in a space efficient manner that takes up less length of table space for the user.

An alternative method of parallel processing test strips using a combination of vertical conveyor and a carousel will now be discussed. This configuration allows a higher number of test strips to be inserted into the conveyor, in a space-efficient manner that takes up less length of table space for the user. Referring now to FIG. 8, the imaging system and mechanism for analyzing test strips 9 will now be discussed. Test strips may be analyzed using optical imaging or fluorescence imaging.

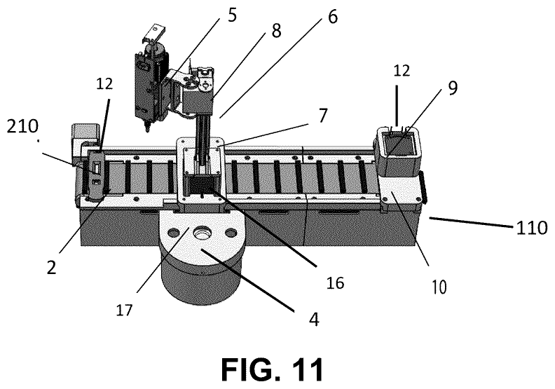

Referencing FIGS. 1, 4, 8, and 11, the imaging system 9 will now be discussed. An imaging system, as disclosed herein, may include a camera to image the test strips, one or more LEDs arranged around the camera to illuminate the test strips, and an optointerruptor to detect the presence of a test strip. To achieve a high-quality imaging system, the components may be selected to have a high resolution and large image area for the sensor, which improves the signal quality and reduces the noise of the raw image. An example of a such high-quality imaging system may include a high-quality, 12 megapixel camera with a fixed 6 mm focal length and 1/2.3″ optical format lens (e.g., Arducam 50 Degree 1/2.3″ M12 Lens with Lens Adapter for Raspberry Pi High Quality Camera), and a ring of twelve 5050 RGBW LEDs (e.g., NeoPixel Ring—12×5050 RGBW LEDs). The camera, one or more LEDs, and/or optointerruptor may be coupled to a microcontroller 11 of the kiosk (e.g., to provide data to and/or receive operational commands from the microcontroller). For example, when the optointerruptor detects a test strip under the imaging system, the optointerruptor may transmit a signal to the microcontroller of the kiosk indicating the presence of a test strip. The microcontroller may then cause the LEDs to turn on (e.g., thereby illuminating the test strip). Thereafter, the camera may capture the image of the test strip and transmit the image to microcontroller for image analysis and/or transmission to external computing devices for image analysis. Camera settings, such as focal distance, International Standards Organization (ISO) setting (i.e., gain), shutter speed, and auto-white balance, may be set by the microcontroller for each image capture in order to ensure repeatability. Alternatively, in some embodiments the camera settings may be pre-programmed into the camera and not modified from image capture to image capture. After the image is captured, the microcontroller may cause the LEDs to turn off.

An example optical system could consist of a high-quality, 12 megapixel camera with a fixed 6 mm focal length and 1/2.3″ lens, and a ring of twelve 5050 RGBW LEDs. Increasing the resolution of the camera and the sensor's image area can improve the signal quality and reduce the noise of the raw image.

An example fluorescent imaging system will now be discussed. The fluorescent imaging system may include an energy source and a reader. The light emitting source consists of a light source with a filter that illuminates the test strip with a specific wavelength of light in the ultraviolet spectrum. The reader consists of an optical sensor and a filter that detects a specific wavelength of light.

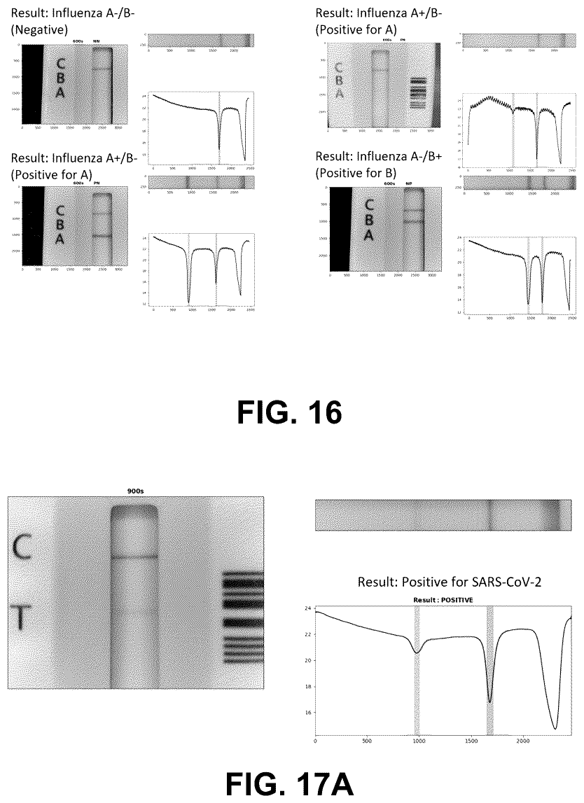

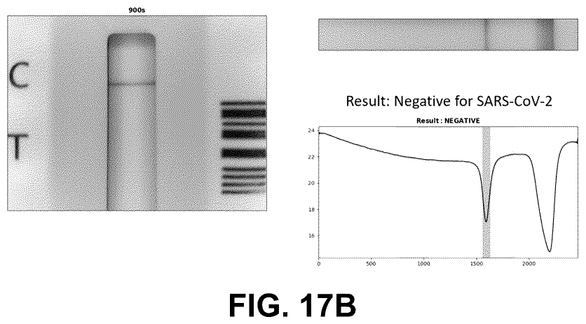

Referring now to FIGS. 16-17, an algorithm for analyzing images of test strips from an optical or fluorescent imaging reader will be discussed. The deterministic image processing algorithm for colorimetric or fluorescent lateral flow immunoassay tests will be discussed. Deterministic geometric image processing algorithms may process optical or fluorescent images of colorimetric or fluorescent qualitative test strip results. The algorithm may read the results by determining the number of test strip lines that appear on the test strip. To determine the number of lines, the algorithm may find the local maxima of the test strip lines, integrate their color values, and compare the integrated result to thresholds.

Referencing now to FIGS. 16-17, a deterministic geometric image processing algorithm for processing and analyzing images of qualitative, lateral flow test strip results will now be further described. FIGS. 16-17 show example results produced by this algorithm for Influenza A+B and SARS-CoV-2 from images of antigen-based, lateral flow immunoassay test strips. After the imaging module 9 images a test strip, the algorithm imports the test strip image and reads the image. The algorithm identifies the boundaries of the test strip by looking at the change in pixel intensity across the width of the image. The algorithm crops the image to within those boundaries. The algorithm applies a Gaussian blur to reduce the noise in the image. The algorithm converts the 2D image into a 1D image by averaging the pixel intensities across each row of the image. The algorithm searches for the control line in the approximate location of the image by comparing the change in pixel intensity against a threshold. If the control line is detected, the algorithm searches for the test result line at an approximate distance from the control line. If the test strip has multiple test result lines, the algorithm search for these test result lines at known approximate distances from the control line. When looking for the control line or the test result line, the algorithm searches across many smaller sections of the image within the approximate location. The algorithm compares the average pixel intensity on either side of the suspected test result line with the suspected test result line's average pixel intensity. If the change in pixel intensity is stronger than a threshold on both sides of the test result line, the algorithm reports the detection of the test result line and a positive result. Otherwise, the algorithm moves to the next section and continues searching within the approximate location. If the algorithm completes the searching process and is unable to find a test result line, the algorithm reports a negative result.

A supervised machine-learning algorithm for colorimetric chemistry-based dipstick tests will be discussed. The supervised machine-learning algorithm will process optical images of colorimetric, semi-quantitative test strip results. The algorithm learns colors from images (e.g., labeled training data) and consists of two components: (i) a color trainer algorithm that identifies the color values for a specific pad and (ii) an image analyzer algorithm that analyzes a test strip and outputs match or not match for each pad. In the color trainer algorithm, 100 images may be taken, and each image may be cropped to 200×200 pixels of a specific color and converted to a hue, saturation, value (HSV) color space. For hue and saturation, the algorithm may identify minimum and maximum values within two standard deviations. The image analyzer algorithm may analyze a test strip and output match or no match for each pad. For example, an image may be taken and converted to an HSV color space. Thereafter, the image analyzer algorithm may apply a mask using the minimum and maximum hue and saturation values, apply one or more morphological filters to remove noise, and apply a threshold on the number of pixels. The scanning or imaging of a unique barcode or QR code on the test strip to connect test results with the unique test ID will now be discussed. As the test strip is imaged by the imaging system, the unique barcode or QR code on the test strip can also be imaged. The detected visual code will be interpreted by the device to determine the unique test ID. Existing libraries such as but not limited to zbar and zxing may be used to process barcodes or QR codes. The test results (e.g., an indication of the presence of one or more patient conditions) will be sent with the unique test ID to an LIS, EHR system, or another HIPAA-compliant database on a cloud server, where it will be matched to the patient ID using the corresponding unique test ID. The test ID may be in a text format. In a 2D barcode, a label may be added.

A method of delivering data and test results will now be discussed. The device may be configured to display results on a screen (e.g., a display of the self-diagnostic kiosk); store and display results in a spreadsheet in a file like a CSV file that can be transferred to and displayed on a separate computer (e.g., a cloud server, a patient computing device, or a physician computing device) or laptop via WIFI, ETHERNET, mobile connectivity, BLUETOOTH, or other means, such as USB storage device; print test results; or send results via Wi-Fi or mobile connectivity or BLUETOOTH to an LIS, EHR system, or a HIPAA-compliant cloud database. The device may display the unique patient ID, unique user or operator ID, unique test ID, test type, test result, and/or time of result on a display, such as a liquid-crystal display (LCD), a light-emitting diode (LED) display, or a cathode ray tube (CRT) monitor.

Another method of delivering data and test results will now be discussed. The device may instead be connected to a companion printer that prints the data, including, but not limited to, the unique patient ID, the unique user or operator ID, the unique test ID, the test type, the test result, and/or the time of result on paper.

Another method of delivering data and test results will now be discussed. The device may send the data, including, but not limited to, the unique patient ID, the unique user or operator ID, the unique test ID, the test type, the test result, and the time of result via WIFI to an LIS or EHR system through point-of-care middleware or directly to a HIPAA-compliant database on a cloud server.

De-identified data collected in an LIS, EHR system, or a HIPAA-compliant database on a cloud server may be regularly sent to public health authorities using Health Level 7 (HL7) messaging or a CSV format. The data may be sent to state and local public health departments through a centralized platform, such as, but not limited to, the Association of Public Health Laboratories' AIMS platform. The data may be submitted through a state or regional Health Information Exchange (HIE) to a state or local health department and then to the Center of Disease Control.

Referring now to FIGS. 2, 6, and 7, a self-service kiosk will be discussed. The user of the self-service kiosk may be a patient, a caregiver, a trained healthcare professional, or a trained non-healthcare professional. A system may include a kiosk 110, a web-based or app-based patient portal, and a self-testing kit 301. In some embodiments, the patient sample may be collected by the patient and placed directly into the analyzer by the patient to be prepared, processed, and analyzed.

A self-service kiosk will be discussed. The kiosk could also be adapted to perform antigen-based, lateral flow COVID-19 and Influenza A+B tests of self-collected nasal fluid samples, saliva samples, or sputum samples. Other sample types are also possible and contemplated herein.

Some embodiments include a touchless analyzer, test strips (e.g., within a multi-test strip cartridge), a self-testing kit with self-collection nasal swabs, sample tube containers pre-filled with buffer extraction solution, and radiofrequency identification (RFID) tags that are integrated with and compatible with the kiosk. Note that the self-testing kit could be configured to contain a saliva or sputum self-collection device instead of a self-collection nasal swab, and the patient would be instructed to collect a saliva or sputum sample instead of a nasal sample.

As an example, schools will be used to illustrate how example embodiments could be deployed and used, but a similar deployment and usage model and principles equally apply to other community settings. The term “students” and “patients” will be used synonymously. As an illustration, self-collected nasal swabs will be used to illustrate how the device functions, but the device would function similarly to process and test other types of samples (e.g., saliva samples or sputum samples). The main difference is the saliva and sputum samples are collected with self-collection devices other than nasal swabs, but, similar to the nasal samples, the saliva and sputum samples may be transferred into the sample container tube pre-filled with buffer extraction solution.

In this example, students may receive self-testing kits at school. The RFID tag's unique identification code would be associated with the student. The student may be guided by simple images and instructions on the device display to collect their own nasal sample using a swab and a sample tube container inside the kit. The student may then be instructed to insert the sample container, as well as the test strip from the kit, into the device for immediate analysis. The device may then perform the point-of-care COVID-19 test in 15 minutes, and send the results along with the RFID tag's unique identification code in an encrypted, HIPAA-compliant format to a secure web portal. The students and school may then be able to access the web portal (e.g., via a web browser) through a smartphone and/or computer to view the test results.

The self-testing kit may include a sample tube container pre-filled with buffer extraction solution and a self-collection nasal swab. Students may collect their own nasal sample using the swab and container.

The sample tube container may have an RFID tag. Sensing the RFID tag on the sample tube container, the device may automatically activate and open a receptacle door (i.e., port). The sample container holder may automatically extend out towards the student via a linear stage to ensure a touch-free experience. The student may then insert the sample tube container in the sample container holder. The device may then automatically perform a COVID-19 test in 15 minutes.

The device may be able to perform each COVID-19 test in 15 minutes. However, in some embodiments, the device may be able to process 10 or 15 patient samples and test strips simultaneously, increasing the device's throughput. In some embodiments, the device may process a new patient sample every 1.5 minutes or even every 1 minute, reaching steady state in 15 minutes. At steady state, the device may produce a new test result every 1.5 minutes or even every 1 minute. As a result, at steady state, the device may have a patient throughput of 40 patients per hour per device or even 60 patients per hour per device, respectively. It is understood that the above is provided solely as an example and that other numbers of patient samples, processing times, times for producing test results, and/or throughputs are also possible and contemplated herein.

The analyzer in the self-service diagnostic kiosk will now be discussed in greater detail. The analyzer may be a portable electromechanical device with automated mechanisms for biofluid preparation/handling, self-washing and decontamination, sample tube container insertion, test strip insertion, optical imaging, activation of the device via RFID or near-filed communication (NFC)-enabled sample tube container, and connectivity with an LIS, an EHR system, or another HIPPA-compliant database on a cloud server.

A web-based (e.g., browser-based) or app-based patient portal will be described in greater detail. The web-based or app-based patient portal may contain the unique patient ID and display tests that have been ordered by a physical or telehealth clinician. The app-based patient portal may access a smartphone camera and image unique barcodes or QR codes.

The testing kit will now be discussed in greater detail. The testing kit may contain a sample tube container with a RFID or NFC tag and a test strip. The sample tube container may be pre-filled with buffer solution, depending on the test. The sample tube containers may be capped with pierceable and resealable caps. Caps may hold layered pockets with multiple reagents. A self-collection swab may be included for tests requiring a nasal swab, a nasopharyngeal swab, a throat swab, a buccal swab, a vaginal swab, or a fecal swab. The test strip may be enclosed in a cartridge or a card. The test strip and/or test cartridge may contain a unique barcode or QR code that can be linked to the test type and unique test ID.

In some embodiments, a custom cartridge may be used to house test strips. The mechanical design and shape of the custom cartridge may integrate with and hold a broad range of test strips. The custom cartridge mechanical design and shape may be optimized for human factors such that patients may intuitively insert the right side of the test strip into the test strip port without mistake and without touching the device. The housing may have a handle that is on the opposite side of the test strip where the sample is dropped. The handle may be too large to fit into the test strip port, preventing the wrong side of the test strip from being inserted.

A method of testing patients will now be discussed. The user(s) may include a patient, a caregiver, a trained non-healthcare professional, and/or a trained healthcare professional. In a diagnostic kiosk, the user may be guided by simple images and instructions on the display, test kit, or patient portal to collect a patient sample using a sample container (sample tube container, sample collection cup, or other sample container form factors), and, for some tests, a swab that is provided inside the kit. The user may then place the swab into the sample container after collecting the sample.

A method of inserting a sample container containing the patient sample into the kiosk in a contactless manner will now be discussed. The user may be instructed by simple images on the device screen or testing kit to insert the sample container into the analyzer. The device may display images on an LCD, an LED display, or a CRT monitor. Sensing the RFID or NFC tag on the sample container, the device may automatically activate and open the receptacle door.

Another method of inserting the sample container containing patient sample into the kiosk in a contactless manner will now be discussed. The user may be instructed by simple images on the display or test kit to place the sample container on a robotic arm. The robotic arm consists of a lift that can move the sample container up and down, a motor that can grip the sample container to pick up the sample container and drop off the sample container in a specified location within the device, and an arm or claw that can grab and hold the sample container.

The sample container holder will now be discussed in greater detail. The sample container holder may automatically extend out towards the user via a linear stage, a linear conveyance system, or a spring-loaded mechanism. The user may then insert the sample container in the sample container holder. When the user places the sample container in the sample container holder, the holder may sense the container using a load cell, a touch sensor, or an optical sensor and, thereafter, return into the receptacle (e.g., based on a command signal transmitted by a microcontroller of the kiosk).

A method of inserting the test strip from the testing kit will now be discussed. The user may be instructed by simple images and instructions on the device's display to insert the test strip into the device's test strip port. The shape of the test strip port may be optimized to enable an easy-to-insert touch-free experience. The methods of inserting the test strip described throughout this disclosure may be used. As an example, the test strip may be inserted through the test strip insertion port onto a parallel processing mechanism, such as a parallel processing conveyor belt mechanism.

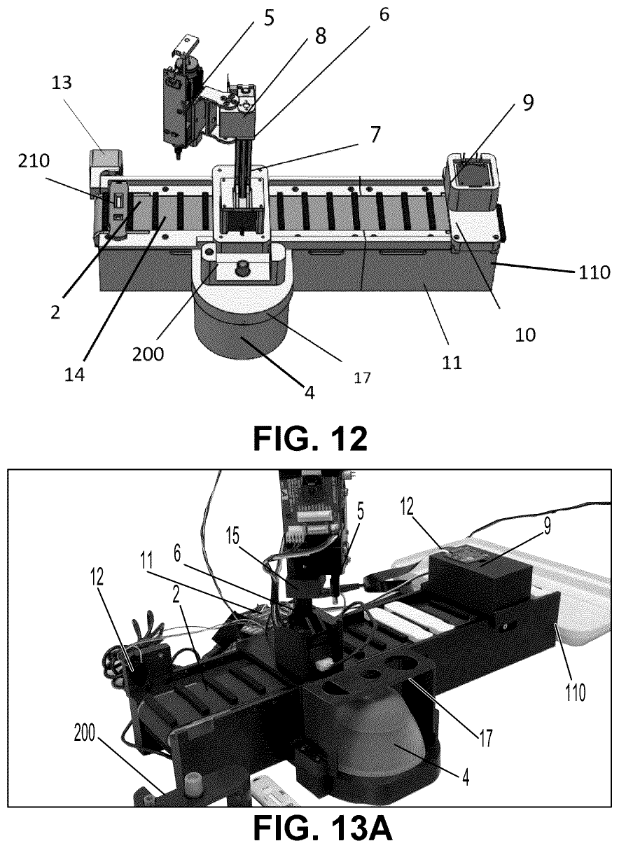

An embodiment of the device that utilizes a test cartridge that houses a plurality of test strips will now be discussed. The analyzer may house an array of cartridges with stacks of multiple test strips inside and/or a range of test strips. The analyzer may automate the test strip usage process with a parallel processing conveyor belt mechanism that pulls test strips from the stacks onto the conveyor belt. Referencing FIG. 12, the analyzer may drop the patient sample via a robotic pipette module 5 onto the test strip while on the conveyor belt, and the conveyor belt may then bring the test strip underneath the imaging system (such as an optical reader (camera) or fluorescent reader) for analysis. After the test strip is analyzed, the conveyor belt may drop the test strip into a waste bin for disposal. The analyzer may also automate the test strip usage process with a linear stage mechanism that pulls test strips from the stacks. The linear stage brings the test strips underneath the robotic pipette module 5 to receive the sample, underneath the test strip analysis sensor for analysis 9, and into the waste bin 3 (FIG. 8) for disposal. A reel-to-reel mechanism may also be used to automate the test strip usage process. The reel-to-reel mechanism holds a reel-to-reel cartridge. The reel-to-reel cartridges may contain multiple test strips overlaid over a reel. The reel-to-reel mechanism brings the test strips underneath the sample volume control and dispense mechanism to receive the sample and bring the test strips underneath the test strip analysis sensor for analysis.

A method of connecting a unique patient ID to a unique test ID will be discussed. A web-based or app-based patient portal may contain the unique patient ID. A smartphone camera accessed by the mobile application may image a unique barcode or QR code on the test strip. The detected visual codes may be analyzed by existing algorithm libraries, such as, but not limited to, zbar and zxing, to determine the test type and unique test ID from the unique barcode or QR code on the test strip. The unique patient ID, test type, and unique test ID may be sent to an LIS, EHR system, or another HIPAA-compliant database on a cloud server database.

An alternative method of connecting a unique patient ID to a unique test ID will be discussed. A web-based or app-based patient portal may contain the unique patient ID. The mobile application on the smartphone may take an image of the barcode or QR code with the smartphone camera. The mobile application may then analyze the barcode or QR code for the unique test ID that connects the barcode or QR code to the unique patient ID. Additionally, the analyzer may sense and analyze the RFID or NFC on the sample tube container or testing kit to determine which test to run and link the test results to a unique test ID. The analyzer may also read (image or scan and then analyze) the barcode or QR code that is on the sample tube container or test strip cassette to determine the unique test ID. The analyzer may also read (image or scan and then analyze) a barcode or QR code on the mobile application to determine a unique patient ID and link the patient ID with the unique test ID.

Alternative methods of connecting a unique patient ID to a unique test ID are also contemplated herein.

A method of automatically mixing the sample with buffer extraction solution for certain tests will now be discussed. The method optimizes the limit of detection of the system by maximizing the volume of sample that is mixed with the buffer extraction solution. For some tests, the device may automatically mix the sample with the buffer extraction solution that is pre-filled in the sample container. For other diagnostic tests (e.g., urine-based test), however, mixing the sample with the buffer extraction may be unnecessary/not performed by the kiosk. The receptacle door may automatically close, and a hidden, durable, and reusable metal cannula may lower and pierce through the pierceable and resealable cap until the cannula reaches the buffer extraction solution surrounding the swab inside the container. The cannula may mix the buffer extraction solution with the sample on the swab (such as nasopharyngeal, nasal, oropharyngeal, or buccal fluid) by aspirating and dispensing the fluids multiple times. The positioning of the cannula may be controlled by optointerrupter(s). Additionally, the entire or part of the sample container may be compressible and elastic. The device may have cams that rotate around the sample container to compress the sample container and the swab inside the container. Cams may be oriented perpendicular to sample vial longitudinal direction, parallel to base of device. Two cams may rotate via separate motors in opposite clockwise rotations, one clockwise and one counterclockwise. Threaded faces of cams may grip and compress a flexible sample vial to extract embedded sample in swab and mix further with sample buffer. The cams may rotate multiple times, and may rotate before, during, and/or after the aspiration and dispense mixing of the fluids by the cannula. The cams may be controlled by motor(s) (stepper or DC) with or without a system of gears. The cams may be controlled by a motor with a system of gears. The cams, motors, and system of gears may be controlled by a microcontroller 11 (FIG. 8) of the kiosk.

Another method of automatically mixing the sample with buffer extraction solution will now be discussed. Cams may be oriented parallel to the sample vial longitudinal direction. Cam may have multiple alternating faces, like in a car engine, or a spiral orientation of protrusions, like in a spiral staircase. Cams may run on a singular motor but with a gear system, so their movement is synchronized.

If there are additional reagents in addition to the buffer extraction solution, the cannula may draw the layers of reagents into the sample container as it pierces through the cap. The cap may contain layers of reagents. The cannula may mix the reagents with the dominant solvent (buffer extraction solution) and the sample on the swab inside the sample container.

A method of extracting samples will now be discussed. The sample mixing and extraction cannula may extract the sample and transport it to a sample volume control and dispense mechanism via tubing and a small pump. The cannula lowers a syringe via linear actuator into sample tube to extract mixed sample buffer. This mechanism may dispense a controlled volume of sample onto the test strip. The mechanism may be comprised of a sample dispensing cannula, a tubing pathway, and a small pump. The tubing pathway and small pump may be connected to the sample extraction cannula in the sample mixing and extraction system described above. After the sample has been mixed with buffer extraction solution (if necessary), the sample extraction cannula may draw the mixed sample fluid from the sample container via the small pump. The small pump may then bring the mixed sample fluid through the tubing pathway to the sample dispensing cannula at a relatively high flow rate to maximize throughput. The small pump may slow down the flow rate as the mixed sample fluid approaches the end of the sample dispensing cannula to control the sample volume. The small pump may rotate slowly in pulses such that the sample fluid exits the sample dispensing cannula in controlled numbers of droplets. A controlled number of droplets, and hence a fixed volume of fluid, is dropped onto a test strip through the sample dispensing mechanism. The mechanism may be modified (e.g., based on one or more control signals transmitted by a microcontroller 11 (FIG. 8) of the kiosk) to dispense different amounts of samples onto test strips, depending on the test. Further, in some embodiments, the diameter of the tubing may be adjusted and/or a reducer nozzle may be used, depending on the test, to alter dispensed volumes of sample. The diameter of the tubing may be made thinner to achieve higher accuracy and precision in controlling the sample volume that is dispensed onto the test strip. The small pump may be a peristaltic pump or a microperistaltic pump.

The mechanism may also be a y-shaped tubing apparatus controlled by a three-way solenoid valve and a software program. The tubing pathway bifurcates into two tubing pathways. The first pathway collects a fixed volume of sample and drops the sample onto each test strip as droplets. The second pathway allows excess volume of the sample not needed for the analysis to flow through. The mechanism may be modified to dispense different amounts of samples onto test strips, depending on the test. The diameter of the tubing may be adjusted, and a reducer nozzle may be used, depending on the test.

A method of disposing of the used sample container will now be discussed. After extracting the mixed sample, the sample mixing and extraction cannula may raise and leave the container and resealable cap. The cap may reseal itself when the cannula leaves. The cap may have a pierceable layer (e.g., metallic foil, such as aluminum foil; clear plastic polymer wrap; etc.) with a duck bill valve to reseal itself. The sample container holder may extend out and rotate upside down to drop the used sample container into a waste bin. Alternatively, robotic arm may grab the sample container and drop it into a waste bin.

An alternative method of disposing of the used sample container will now discussed. The receptacle platform may extend back out towards the user, and the user may remove the container from the sample container holder. The sample container holder may return into the receptacle, and the receptacle door may close.

A method of cleaning the device to prevent cross-contamination of patient samples will now be discussed. The device may begin wash cycles with a wash buffer and water. The sample mixing and extraction cannula may lower itself into the empty sample container holder. The sample container holder may be connected via tubing and small pumps to a wash buffer container and water container, which may be hidden beneath or within the kiosk. The containers may be modularly connected to the device via quick-release connectors on the tubing. The device may fill the sample container holder with a wash buffer, followed by water, in subsequent wash cycle(s). The outer and inner surface of the sample mixing and extraction cannula that entered the container may be covered by a wash buffer, followed by water, a process that washes and decontaminates the cannula. The wash buffer may be a diluted sodium hypochlorite (bleach) solution. The water may be purified water, tap water, distilled water, or deionized water. Alternatively, a buffer solution, such as a salt buffer solution (e.g., a phosphate buffer solution), may be used instead of water to minimize non-specific binding.

The sample mixing and extraction cannula may draw the wash buffer and water. The wash buffer and water may be transported through tubing, cannula, or device surfaces that touched the sample. The wash buffer may denature residual biomarkers on surfaces, and the water may remove any residual wash buffer. Multiple wash cycles may occur to prevent carryover from different patient samples touching the same surfaces, ensuring no cross-contamination and enabling clinical-grade accuracy. The wash fluids may be transported into a waste container, which may be hidden underneath or within the kiosk.

After each analysis cycle, the kiosk may wash itself with a diluted sodium hypochlorite (bleach) solution, which may denature and remove any residual biomarkers, viruses or viral particulates, bacteria or bacterial particulates, cells or cell particulates, or other sample components or constituents or particulates that are on the cannula, tubing walls, cannula walls, or device surfaces without damaging them. The kiosk may then wash away the sodium hypochlorite with water so that future biomarker results at the limit of detection and chemical sensitivity are not impacted by residual sodium hypochlorite.

The washing sub-system may be connected modularly via tubing and quick release connectors to wash buffer/water sources and a waste destination. The used washing solutions may be routed into a waste bin, which may be hidden underneath the kiosk. The wash buffer and wash water sources may be stored in containers, which may be hidden underneath the kiosk. Waste containers may be disposed of through standard hazardous waste disposal practices.

The kiosk may be placed near a sink and modularly connected via tubing and quick release connectors to the sink. The waste fluids may be routed into the sink for disposal. Additionally, the device may draw water from the sink to wash the device. The device may also use this water to self-dilute a concentrated bleach solution to self-wash itself with. In this permutation of the kiosk, the need for hidden containers to hold wash buffer, wash water, and waste fluids may be substantially reduced and/or eliminated altogether. The analyzer may also be connected to a deionized water source.

Kiosk devices disclosed herein may make rapid testing safer and more accessible. A method of diagnostic testing will now be discussed. The device may be deployed in minimally staffed or unstaffed retail stores or pharmacies, clinics, and community-based settings, including, but not limited to, walk-in and drive-in settings. Patients or caregivers may be directed to use the self-service kiosk device by remote clinician via telehealth or a physical clinician. Testing kits may be procured by patients and caregivers directly on-site from a retail store or pharmacy, a clinic or another community-based setting, dispensed from an on-site vending machine, or sold online and delivered direct to the consumer by mail. In another workflow, a trained healthcare professional or non-healthcare professional that is on-site may collect the patient sample and initiate testing using the device instead of the patient or caregiver. The kiosk may automatically perform a point-of-care test, and send the results along with the unique identification code from the RFID and/or NFC tag and/or barcode(s) and/or QR code(s) to a remote clinician on an EHR-integrated telehealth platform. Remote clinicians may review and release the test results and guide the patient with the next steps in clinical management.

The devices disclosed herein may also be used as part of novel healthcare delivery model. A method of delivering clinical interventions will now be discussed. Remote clinicians (e.g., via telehealth) or physical clinicians may pre-authorize certain interventions based on test results from the self-service kiosk, including, but not limited to, filling prescriptions, placing an order for a central laboratory test, or recommending over-the-counter medications or other items via text, email, or patient portal, scheduling follow-up appointments via text, email, or patient portal, referring patients to a specialist via text, email, or patient portal. The test result may automatically trigger a pre-authorized action, directing the next step of care. An example is shown where a prescription is automatically filled at a pharmacy after a patient uses the self-service kiosk.

FIG. 2 is an illustration of a self-service diagnostic kiosk. The self-service diagnostic kiosk may be utilized in virtual worksite clinics, for example. At worksites, the self-service diagnostic kiosk may be utilized in private office rooms or wellness rooms, for example. Further, the self-service diagnostic kiosk may perform diagnostics before, during, or after telehealth visits.

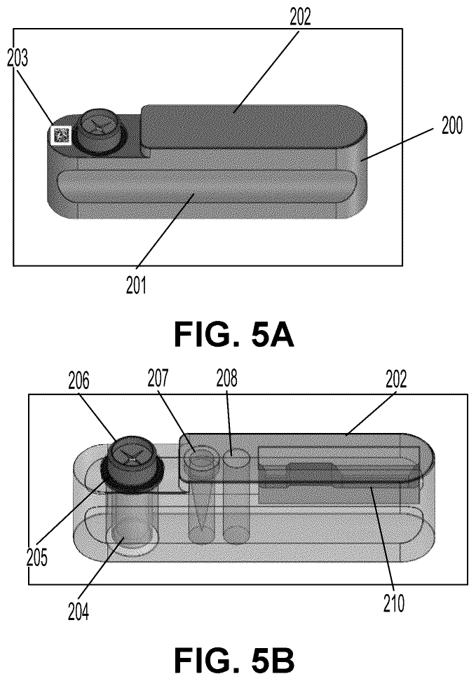

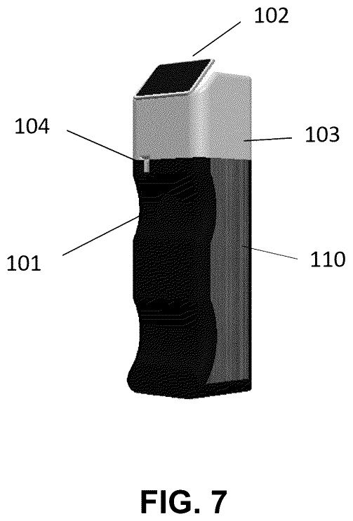

Referring now to FIG. 5, the test cartridge 200 of the self-service diagnostic kiosk will now be described. The test cartridge may include a shell 201 with ergonomic design for easy holding and loading into the kiosk with one hand, partially or fully made of moisture-impermeable material. The test cartridge houses a test strip 210, and the test cartridge may fully enclose the test strip with moisture-impermeable material (such as aluminum foil, aluminum sealing foil, glass, or a plastic polymer) to preserve the test strip within the test cartridge and protect the test strip against moisture from the fluids in the test cartridge chambers or the environment. The test cartridge may also have a moisture impermeable seal layer 202 (e.g., made of metallic foil, such as aluminum foil or aluminum sealing foil, or clear plastic polymer wrap) above the test strip and other components (e.g., disposable pipette tip 207, chamber with wash buffer 208) configured to preserve the test strip within the test cartridge and protect the test strip against moisture from the fluids in the test cartridge chambers or the environment. Before the kiosk pierces and/or opens the seal layer, the seal layer may also protect all of the components underneath it (e.g., disposable pipette tip 207, chamber with wash buffer 208, test strip 210) from the environment, contaminants in the environment, and/or the user accidentally touching these components with their fingers when handling the test cartridge, thereby preventing cross-contamination of these components. The test cartridge may have a QR code 203, one-dimensional or two-dimensional barcode, or similar barcode label on cartridge that links the test cartridge number with the test result and test order number. The test cartridge can contain a desiccant or multiple desiccants at a location or multiple locations, such as underneath the seal layer, to help preserve the test strip and protect the test strip against moisture. The packaging (e.g., wrapper) for the test cartridge may contain desiccant to help preserve the test strip and protect the test strip against moisture. Further, the kiosk may be configured to read the label/associated barcode using the imaging system 9 (FIG. 8) and/or using a dedicated barcode scanner or QR code scanner at a location such as the test cartridge insertion port 104 (FIG. 7).

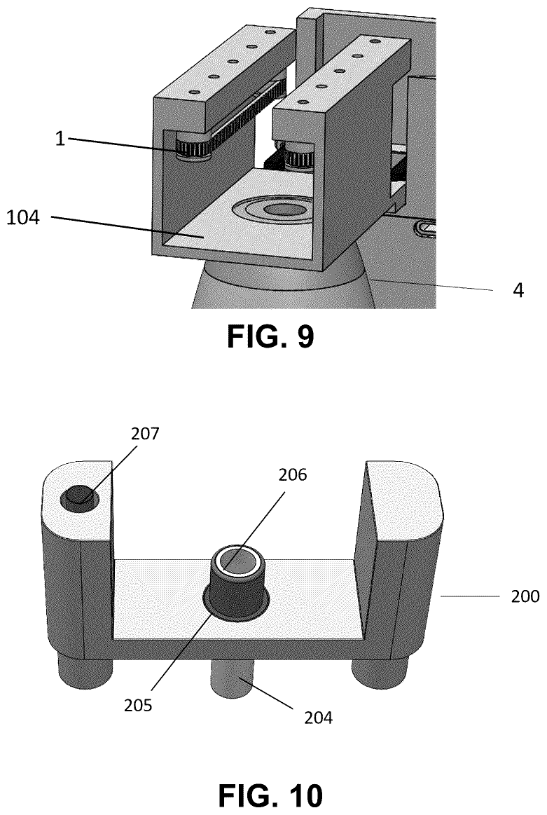

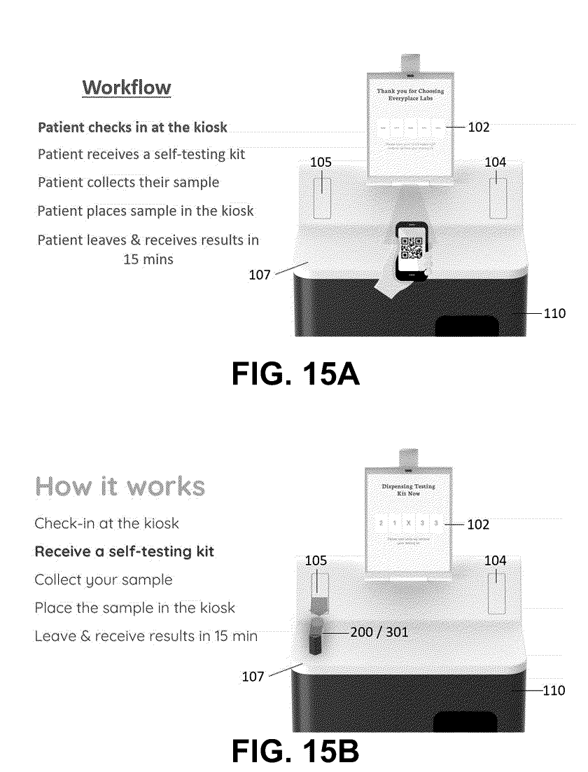

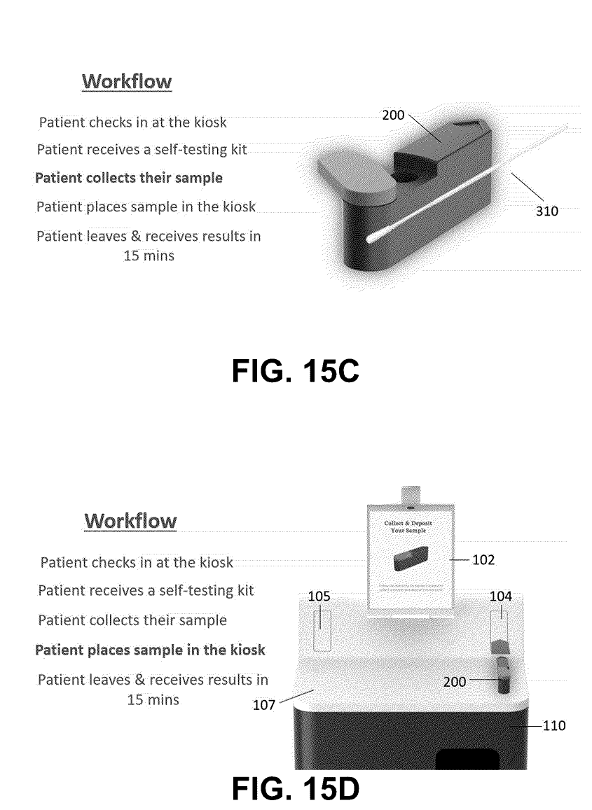

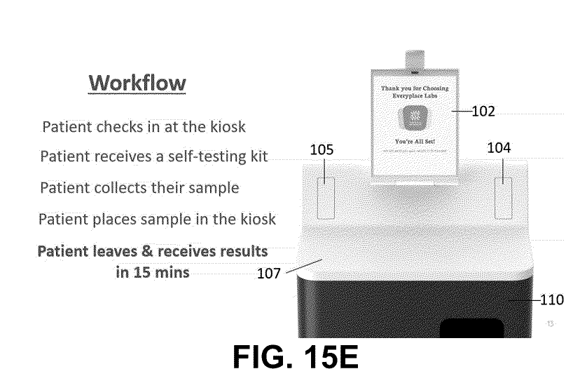

As illustrated in FIG. 5, the test cartridge may have a vial containing buffer extraction solution 204. While the term “vial” is used throughout the disclosure, it is understood that other form factors of containers/chambers containing the buffer extraction solution (and other solutions) are equally possible and are contemplated here. Further, while a single chamber may be described as containing the buffer extraction solution in examples herein, it is understood that, for some diagnostic tests (e.g., tests requiring multiple reagents, such as a test for streptococcal pharyngitis), the test cartridge may contain multiple chambers housing multiple reagents/solutions used for the diagnostic test. The patient may place their sample (e.g, nasal fluid sample on nasal swab 310 (FIG. 15C)) into this vial containing buffer extraction solution. As illustrated in FIGS. 8-9, the kiosk's vortex mixer 4 may vibrate to mix the buffer extraction solution with the patient sample (e.g. nasal sample) to release the viral antigen protein in the patient sample from the viral shell. As illustrated in FIG. 5, this vial has a self-scaling cap (e.g., septum cap) 206, which allows a disposable pipette tip 207 to enter the vial to aspirate a fixed volume of the mixed sample. When the pipette tip enters the vial 204, the pressure from the tip pushes and/or pierces open the self-sealing layer of the cap, which allows the tip to enter the vial. When the pipette tip leaves the vial, the cap's self-scaling layer seals itself.

The self-sealing cap may also allow a pipette tip later to dispense a wash buffer 208 into the vial to convert the regulated medical waste (e.g. patient sample, nasal sample, nasal fluid, etc.) inside into normal municipal waste by chemically decontaminating the potentially infectious contents (e.g., viruses, bacteria, and other pathogens) and patient fluids inside the vial. The self-sealing cap may also limit the amount of aerosolized sample that can escape the vial, minimizing cross-contamination potential from different patients using the same kiosk.

The test cartridge may have a flexible o-ring 205 that holds the vial (with buffer extraction solution) tightly to the cartridge while allowing the vial to vibrate within the cartridge around the free space surrounding the vial during vortex mixing (e.g., using a vortex mixer of the kiosk).

The test cartridge may contain a disposable pipette tip 207 that the kiosk's robotic pipette module picks up to aspirate, dispense, and transport fluids (e.g. the patient sample mixed with buffer extraction solution and/or a wash buffer solution) to different components within the same test cartridge without cross-contamination from different patients who use the same kiosk. The robotic pipette module may return the disposable pipette tip to the original location of the disposable pipette tip within the test cartridge for disposal before the kiosk disposes of the entire test cartridge into the waste bin.

As illustrated in FIG. 5, the test cartridge may have a vial or well or chamber with wash buffer (e.g., diluted bleach) 208 that the kiosk's robotic pipette module uses to decontaminate and convert the liquid-based regulated medical waste (e.g. patient nasal fluid sample) in the test cartridge into normal municipal waste. Per federal and state regulations for proper disposal of regulated medical waste, liquid-based regulated medical waste (e.g., nasal fluid samples, saliva samples, urine samples, blood samples from humans) can be converted into standard municipal waste by chemically decontaminating the liquid-based medical waste with a decontamination chemical solution (wash buffer), such as diluted bleach. The decontamination solution (wash buffer decontaminates the potentially infectious contents (e.g., viruses, bacteria, and other pathogens) in fluid samples from patients (liquid-based regulated medical waste). When the liquid-based regulated medical waste has been converted into standard municipal waste, the converted medical waste can be disposed of like normal municipal trash found in non-healthcare community settings or offices (such as paper), without any special treatment, processes, or training. The converted medical waste can also be dumped down the drain. As a result, converting the patient sample in the test cartridge into standard municipal waste allows non-medical settings in the community to handle disposal of used test cartridges like normal municipal trash without the need for specialized vendors to pick up and ship the medical waste off-site for specialized treatment, conversion, and decontamination, and the need to train staff on how to handle and dispose of regulated medical waste. The vial/well/chamber may have a self-sealing cap on top of it or a seal layer 202/206 on top of it to seal the wash buffer within the vial/well. The “wash buffer” may also be known as and referred to as “medical waste decontamination solution.”

Referencing to FIG. 5, the test cartridge contains a test strip 210 (e.g., lateral flow immunoassay/aptamer assay or clinical chemistry test strip, antigen-based lateral flow immunoassay). Where test strips are described throughout this disclosed, it is understood that other testing containers, formats, or modalities could equally be used and are contemplated herein. For example, a chamber used for a liquid-reagent-based assay or a microfluidic liquid-reagent-based assay may be used in addition to or instead of a test strip. Such chambers may be used when the patient sample is a blood sample, for instance. The kiosk's robotic pipette module may dispense the patient sample and/or mixed patient sample/buffer extraction solution onto the test strip for analysis of the patient sample. The walls surrounding the test strip can be made of moisture impermeable material (e.g., sealing foil/scaling film) to protect the test strip against moisture from the environment and moisture from the vial with buffer extraction solution and the vial with wash buffer.

The test cartridge may also contain a cutout window at the top of the test cartridge. Such a cutout window may be protected by a seal layer 202. The seal layer may protect the test strip underneath the seal layer until the test strip is to be used. This may protect the test strip against moisture from the environment, moisture from the vial with buffer extraction solution, and/or moisture from the vial with wash buffer. The kiosk's robotic pipette module may pierce and/or open the seal layer, segments of the seal layer, and/or all of the seal layer to access the components underneath the seal layer as they are needed. Parts or all of the seal layer may be made of transparent material, so that the viewing window above the test strip results zone can be imaged by the kiosk's imaging module, without the kiosk needing to open the seal layer.

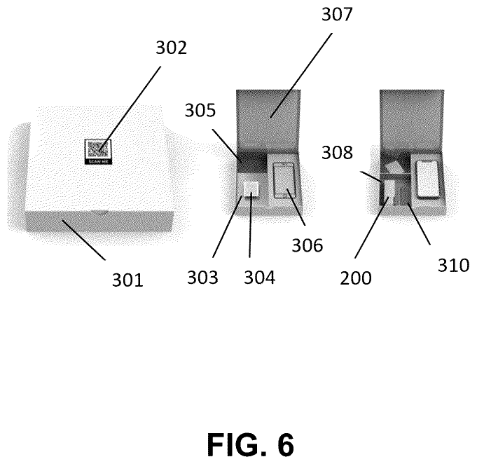

Referencing FIG. 6, a self-collection kit of an embodiment of the self-service diagnostic kiosk will now be described. The self-collection kit may be a disposable box 301 (e.g., cardboard box) with a QR code or barcode label 302. The box may be a literature-style mailer box, and the box may be sterilized. The self-collection kit can have a gating-step (e.g., a peelable layer 303) that holds pack(s) of sanitization wipe(s) 304 on top of it (or potentially adhered onto it). The patient may use the cleaning wipe(s) 304 to clean their hands, phones, and/or other surfaces such as a public, community table the kit may be placed upon. This cleaning process may clean the self-collection environment, preventing cross-contamination during self-sample collection and spread of viruses and bacteria from different patients self-collecting in the same environment.

The peelable layer may be above a compartment 308 that holds the test cartridge 200 and sample self-collection method/tool (e.g., self-collection nasal swab) 310. When the peelable layer with sanitization wipes is above the compartment, the peelable layer may serve as a gating step and prompt patients to clean their hands, phones, and/or environment before accessing the test cartridge and self-collection method/tool in the compartment. The self-collection method/tool can be a self-collection nasal swab, which allows the patient to self-collect nasal fluid from both of their nostrils.

The self-collection kit may also include a waste compartment 305 that the patient can use to dispose of waste and/or wrappers for the test cartridge and sample self-collection method/tool into. The sanitizer wipes may be disposed of in the waste compartment, as well. The kit can have a surface 306 for the patient to place their phone on during the sample self-collection process. The patient can access and refer to instructions on their phone to guide themselves through actions, such as navigating the self-testing kit, self-sanitizing their hands, phones, and/or environment, self-collecting their sample, and using the kiosk. The self-collection kit can also have written instructions 307 to guide the patient through parts of or the entire the self-testing process, including actions such as how to navigate the self-testing kit, self-sanitize their hands, phones, and/or environment, self-collect their sample, and use the kiosk.

Referencing FIG. 7, the externals of the kiosk will now be described. The kiosk may include a device shell 101. The device shell can be modular. Some or all of the device shell can be treated with an antimicrobial coating layer 103 to prevent cross-contamination and transmission of disease from touching of the kiosk by different patients that may inadvertently occur. Additionally or alternatively, one or more of the testing components internal to the kiosk can be treated with an antimicrobial coating layer. The kiosk may include a display 102 that displays instructions to guide the patient through parts of or the entire self-testing process, including actions such as how to self-collect their nasal sample and/or use the kiosk. The patient can navigate through the instructions on the display by interacting with voice or motion sensing or other types of sensing of the kiosk, so that the patient does not need to touch the screen. The display can also be coated with antimicrobial coating and be a touch screen, allowing the patient to navigate the instructions by touching the touch screen without cross-contamination and spread of disease occurring from different patients interacting with the screen. The kiosk also has a test cartridge insertion port 104 that enables the patient to insert the cartridge in a contactless manner, preventing cross-contamination and spread of disease from different patients using the kiosk.

Referencing FIG. 8, the internals of the kiosk will now be described. The contactless test cartridge insertion port may have input conveyor(s) 1 (e.g., one or more roller conveyors). Alternatively, the input conveyor(s) 1 (FIG. 9) may include one or more timing belt conveyor(s) or other types of conveyors instead of the roller conveyors where the input conveyors 1 in the form of roller conveyors are located in FIG. 8. As a patient/user inserts the test cartridge into the port, the input conveyors(s) 1 may rotate to bring the cartridge fully into the port, allowing a contactless loading experience for the patient/user, and then continue to rotate to transport the test cartridge to the vortex mixer 4 for sample mixing as shown in FIG. 8. After mixing, the input conveyor(s) 1 may rotate to bring and load the test cartridge onto the conveyor belt 2 as shown in FIG. 8. The conveyors may be controlled by command signals from the microcontroller 11 of the kiosk.

Referring to FIG. 8, the conveyor belt 2 may hold, convey, and/or transport a singular or multiple test cartridge(s) to different locations (e.g., a first location, a second location, a third location, etc.) within the kiosk throughout the testing/analysis/waste conversion and disposal process and allow for parallel processing of multiple test cartridges from multiple patients. The conveyor belt may have multiple slots 14 (FIGS. 8 and 12) across it to hold multiple test cartridges and allow for parallel processing. The movement of the conveyor belt may be controlled by a motor (e.g. stepper motor) 13 (FIG. 12), and the movement of the conveyor belt and the motor by command signals from the microcontroller 11 of the kiosk (FIG. 8). An example conveyor belt that could be used would be an AS Conveyor Systems Type20 miniature conveyor belt. An example stepper motor that could be used would be a NEMA 17 stepper motor. The kiosk may also include an internal waste receptacle (e.g., waste bin) 3 (FIG. 8) that collects and stores used test cartridges for disposal after the test result has been analyzed by the kiosk and the medical waste inside the cartridges has been converted into normal municipal waste by the kiosk.

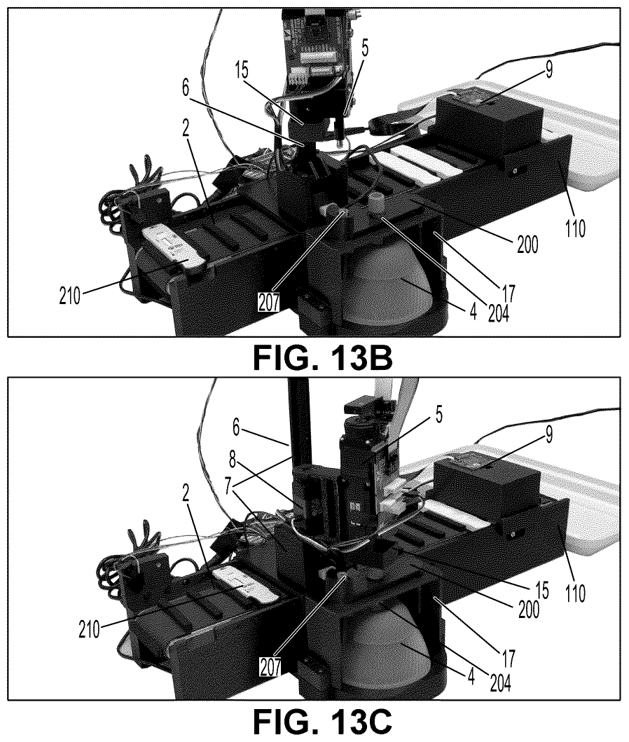

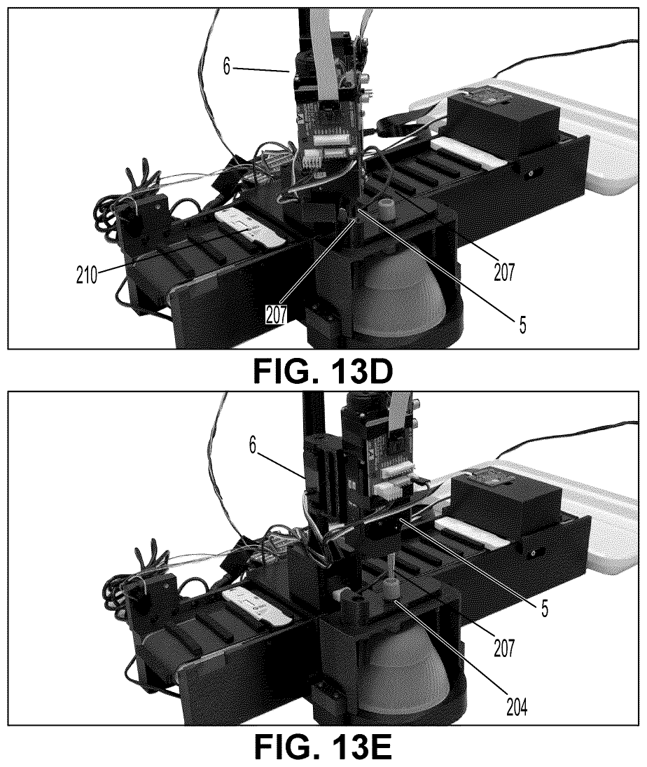

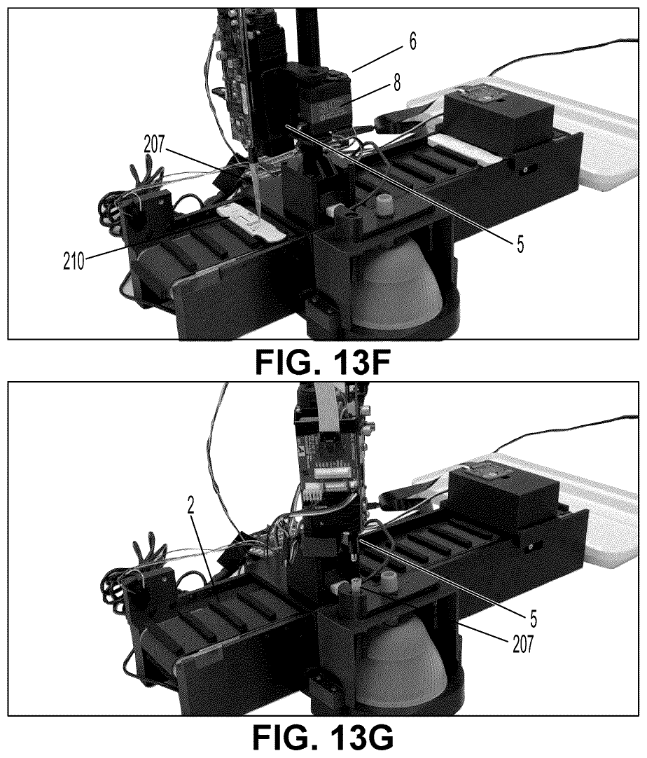

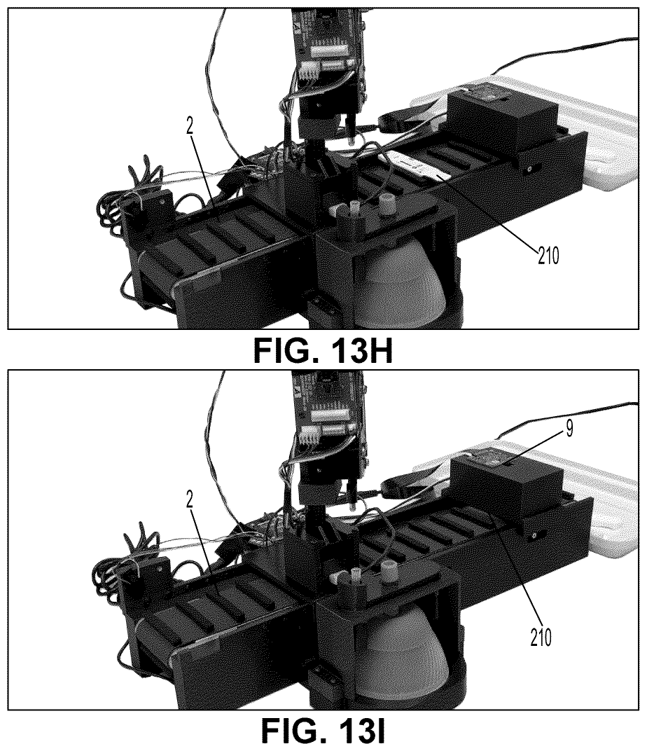

Referring to FIGS. 8 and 9, the kiosk may also include a vortex mixer 4 that vibrates (e.g., due to a command from a microcontroller of the kiosk and/or when a force is applied to the vortex mixer) and mixes the patient sample (e.g., nasal sample on nasal swab, saliva) with buffer extraction solution, which may release the viral antigen protein in the patient sample from the viral shell. The vibration can be continuous or in pulses. The vortex mixer may include an electric motor with both a shaft and a rubber piece mounted slightly off-center. When the motor is running, the rubber piece may oscillate in a circular motion at a high rate. The motor speed may be variable or fixed. The operation may be continuous or in pulses. Alternatively, the operation may only occur when the rubber piece receives an external force (e.g., when the rubber piece is pushed down upon) (FIG. 13C). In some embodiments, the vortex mixer can directly interface with and vibrate the test cartridge's vial that holds buffer extraction solution and the patient sample (FIG. 13C). The vortex mixer can also directly interface with and vibrate the entire test cartridge, thereby also vibrating the test cartridge's vial that holds buffer extraction solution and the patient sample. An example vortex mixer that could be used is a LabGenius MI0101001 mini-vortex mixer with 3000 revolutions per minute.

Referring to FIG. 8, the kiosk may further include a robotic pipette module 5, whose positioning is controlled and moved linearly and rotationally by a motion system 6. The robotic pipette module 5 picks up and utilizes the disposable pipette tip 207 (FIGS. 5 and 10) that comes with the test cartridge to aspirate, dispense, and transport fixed and controlled volumes of fluids from and to different components within the test cartridge (e.g., mixed patient sample with buffer extraction solution, wash buffer). The robotic pipette module may eject the pipette tip back to its original location in the test cartridge for disposal.

The robotic pipette module (e.g., including an associated motion system) may be controlled by control signals from the kiosk's microcontroller 11 to position the robotic pipette module and aspirate and dispense fixed volume of fluids. The microcontroller may receive signals from optointerrupters on the positioning of the robotic pipette module and interpret these signals to control the motion system. The microcontroller may receive signals from level sensors in the robotic pipette module on the volume of fluid that has been aspirated within the pipette tip in order to precisely control the volume that is aspirated. The robotic pipette module may be controlled electronically by interfacing with a computer or a microcontroller via USB or Ethernet using a RS232, a RS485, or a CAN (controlled area network) interface. The robotic pipette module may communicate with the microcontroller in the kiosk using protocols including Data Terminal protocol and CAN bus. Further, the robotic pipette module may include an air displacement pump (e.g., syringe pump/plunger controlled by high-resolution stepper motor), an encoder, and an integrated controller for extremely precise fluid control. The robotic pipette module may be fully self-contained and incorporate various sensors to detect the presence of a pipette tip, detect fluid levels within a pipette tip (e.g., liquid level sensor—pressure and capacitive), and/or monitor, report, and control all activity. The robotic pipette module may include a pipette tip ejector, a plastic piece that pushes the pipette tip off of the robotic pipette module when the microcontroller sends it a command signal to do so. Example robotic pipette modules that could be used include the TriContinent Air-Z Mini or Air-Z Premier air displacement pipette pump.

The robotic pipette module may include a motion system 6 that allows for cartesian/linear movement (e.g., along the x-direction, y-direction, and/or and z-direction) and rotational/polar movement (e.g., along the theta direction and/or the phi direction). This motion system may allow the robotic pipette module to move to and access different components within the test cartridge, and transverse across different sections of the conveyor belt 2 and the kiosk 110. In an example embodiment of the motion system, the robotic pipette module may be connected to the motion system through an arm, and the motion system can be comprised of a linear actuator 7 for vertical linear motion and a servo motor 8 for horizontal rotational/polar motion. An example linear actuator that could be used would be a Haydon Kerk Size 17 43 mm Double Stack linear actuator, and an example servo motor that could be used would be an Uctronics RDS3115 digital servo. In addition, the robotic pipette module may include an automated air displacement pipette pump that is controlled by the motion system described above.

The kiosk may include a high-quality imaging system (e.g., an optical or fluorescent imaging module) 9, such as a high-quality camera that images the test strip within the test cartridge. For example, the imaging system may image a test strip's test result line(s) and control line for analysis by the kiosk. A backend image processing algorithm (e.g., executed by a microcontroller of the kiosk or a HIPAA-compliant cloud database that the kiosk sends the images to) may determine the test result (e.g., the presence or absence of one or more patient conditions) using the image of the test strip. Additionally or alternatively, the imaging system may image a QR code or barcode label 203 (FIG. 5) on the test cartridge to be read by the kiosk. A backend image processing algorithm (e.g., executed by a microcontroller of the kiosk or a HIPAA-compliant cloud database that the kiosk sends the images to) may determine the test cartridge number from an image of the label. Further, the test cartridge number may be linked (e.g., by the microcontroller of the kiosk) to the test result(s) and/or test order number and/or self-testing kit number. The imaging system may be within an enclosure 10 that controls lighting and/or prevents non-desired background light from reaching the test strip during imaging. The camera may interface with the microcontroller via USB or Camera Serial Interface 2.

In some embodiments, the imaging system can contain light-emitting diodes and a camera for capturing images. Alternatively, the imaging system can include a fluorescent imaging module or a hybrid optical imaging/fluorescent imaging module. In a fluorescent imaging module, the LEDs can include one or more ultraviolet spectrum LEDs.

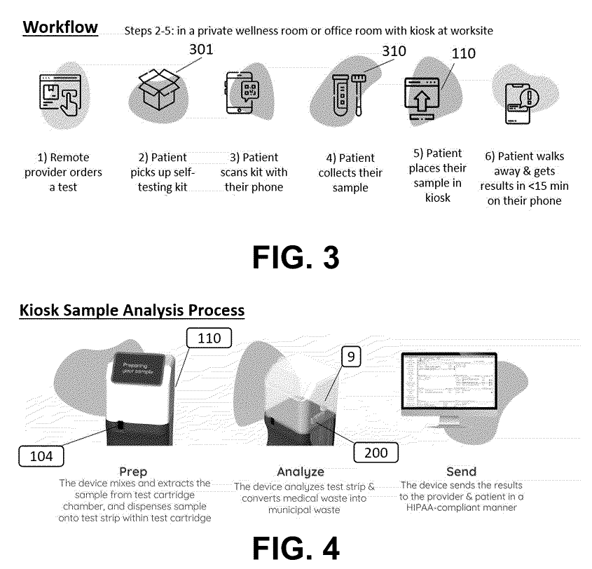

Referencing FIGS. 3, 4, and 6-8, a method of the patient/primary user interacting with the embodiment of the self-service diagnostic kiosk and an associated workflow will be described. In this method, the patient/primary user may be an employee working on-site at a worksite. If the employee feels ill (e.g., with flu-like symptoms) while on-site at the worksite, the employee/patient can consult a remote healthcare provider through a telehealth platform.

The remote healthcare provider may then order a diagnostic test associated with the self-service diagnostic kiosk for the employee/patient, and send a test order number to the patient. The test order and test order number can be sent to the employee/patient's phone (e.g., via a mobile application or a web browser). The remote provider may additionally send instructions to the patient's phone used to: (i) guide the patient to the location in the worksite where a self-testing kit for the diagnostic test can be retrieved and/or (ii) how to find and/or use the self-service diagnostic kiosk. The instructions may also guide the patient on how to use the self-testing kit and/or how to prepare for the self-administered lab test.

The employee/patient may follow the instructions and go to a private wellness room or private office within the worksite. This private room may contain the self-service diagnostic kiosk and provide privacy for the employee/patient to self-test. There or nearby, the employee/patient may pick up a self-testing kit. A medical office assistant may be present at or nearby the private room to facilitate the process of the employee/patient picking up the self-testing kit.

The employee/patient may place the self-testing kit on a table in the private room nearby the self-service diagnostic kiosk. The employee/patient may then scan the self-testing kit's QR code or barcode with their phone (e.g., using an integrated camera of their phone) to link the self-testing kit ID number and test cartridge ID number with the test order number. Following instructions on their phone and/or in the self-testing kit, the patient may self-collect their sample. In the sample self-collection process, the patient may open the self-testing kit and see a gating layer (i.e., peelable layer) with sanitizing wipe(s). The instructions on the kit/their phone may prompt them to clean their hands, their phone, and/or the table surface that the kit is on prior to continuing. This step may reduce cross-contamination potential between patients, which reduces the likelihood of false positives for tests and reduces the spread of surface-borne diseases between patients. The patient may then place the used wipe(s) and wrappers in the waste partition.

After the sanitization process, the patient may then lift the gating layer (i.e., peelable layer). The patient may next open the wrapper for the self-collection tool/method (e.g., self-collection nasal swab) and the test cartridge. The patient may then place the wrapper in the waste partition. The patient may then self-collect their sample (e.g. nasal sample) with the self-collection tool/method/nasal swab. In some embodiments, the patient may self-collect a nasal fluid sample from their nostrils using a self-collection nasal swab.

The patient may place their self-collected sample in the test cartridge. In some embodiments, the patient may open a cap on the test cartridge, place the nasal swab holding their nasal fluid sample in the vial underneath the cap, break the swab handle off of the swab tip by pulling the handle at the handle's breakpoint against the vial, and secure/close the cap back onto the vial.

In various embodiments, the patient could self-collect various types of samples, such as saliva, fingerstick/fingerprick blood, venous blood, throat swabs, vaginal swabs, etc. using self-collection tools and methods designed for these types of samples. The patient may go to another room with more privacy, such as a restroom, to self-collect samples such as urine and vaginal swabs, which would require additional privacy. The patient may place the self-collected sample into the test cartridge, which would have an alternative form factor custom to each type of sample.

The patient may insert/place the test cartridge holding their sample into the self-service diagnostic kiosk in the private room by inserting the test cartridge into the kiosk's test cartridge insertion port. After inserting the sample, the patient may receive the test results on their phone (e.g., via a mobile application or a web browser) within 15 minutes.

Referencing FIGS. 4, 8, and 9, a process will be described for an automated diagnostic testing process that occurs within the kiosk for the embodiment of the self-service diagnostic kiosk.

After self-collecting a sample, the patient may load a test cartridge into a test cartridge insertion port of the kiosk. As the cartridge is inserted into the port, a series of input conveyors (e.g., roller conveyors) or a plurality of input conveyors (e.g., timing belt conveyors) inside the port may engage to bring the test cartridge fully into the port. The patient may then let go of the cartridge as the conveyors bring it fully into the port, allowing this insertion process to be a contactless process. The patient may not need to touch the kiosk, which improves willingness to use the kiosk and minimizes cross-contamination and spread of disease across different patients.

The input conveyors may bring the cartridge onto a vortex mixer inside the port. The vortex mixer may directly interface with and vibrate the cartridge's vial holding the buffer extraction solution and patient sample (e.g., nasal fluid sample on nasal swab) to mix the patient sample with the buffer extraction solution within the vial to generate a mixture. Alternatively, the vortex mixer may directly interface with the cartridge shell and vibrate the entire cartridge, thereby indirectly vibrating the cartridge's vial holding the buffer extraction solution and patient sample. The mixing process may elute the nasal fluid sample from the nasal swab into the buffer extraction solution with high efficiency, and mix the eluted nasal fluid sample with the buffer extraction solution. The buffer extraction solution may cause the viral antigen proteins in the nasal fluid sample to be released (extracted) from the viral shell with high efficiency. While the term “buffer extraction solution” is used herein, it is understood that the term is to be construed broadly to include an fluid (e.g., including or not including reagents) used to elute and extract a sample for dispensing onto a testing device (e.g., a test strip).

After the mixing process, the test strip insertion port's conveyor(s) may load the test cartridge onto a test cartridge slot on a conveyor belt perpendicular (or in series) to the test cartridge insertion port. The conveyor belt may transport the test cartridge to different stations within the kiosk throughout the testing/analysis/waste conversion and disposal process (e.g., to a first location within the kiosk, a second location within the kiosk, a third location in the kiosk, etc.).

The belt conveyor may transport the test cartridge to the robotic pipette module (e.g. from a first location in the kiosk to a second location in the kiosk). The movement of the robotic pipette module may be controlled by a robotic motion system (e.g., based on one or more control signals received from the microcontroller of the kiosk) that gives the robotic pipette module linear and/or rotational degrees of motion. The robotic pipette module may pierce/open the seal layer on the test cartridge so that the disposable pipette tip, test strip, and/or wash buffer vial in the test cartridge are accessible to the robotic pipette module. The robotic pipette module may pick up the disposable pipette tip within the test cartridge. The robotic pipette module may enter the test cartridge's vial holding the mixed buffer extraction solution and patient sample with the disposable pipette through the vial's self-sealing cap. The robotic pipette module may aspirate a fixed volume of the mixed sample in the vial into the pipette tip. The robotic pipette module may leave the vial, and the vial's cap may self-seal when the robotic pipette module leaves. The robotic pipette module may dispense the mixed sample in the pipette tip onto the test strip in the test cartridge.

The test strip may develop over a pre-specified period of time (e.g., 5-15 minutes) that is dependent on the test strip type within the test cartridge (Influenza A+B, Covid-19, multiplex Influenza A+B/Covid-19, general urinalysis, drug, diabetes, chronic diseases, etc.). When the appropriate development time has been reached, the conveyor belt may bring the test cartridge underneath the imaging system (e.g., from a second location in the kiosk to a third location in the kiosk), and the imaging system may image the test strip. The kiosk may analyze the image using an image processing algorithm to determine the test strip result.

When an image of the test strip is being captured, the imaging system may also capture an image of the barcode/QR code label on the test cartridge to serve as extra validation to confirm the test cartridge ID number/test type. The image of the barcode/QR code label may be the same image as the image of the test strip and/or a different image captured by the imaging system, in various embodiments. The test cartridge ID number/test type may be linked with the test result(s), test kit ID number (also known as self-testing kit ID number), and/or test order number. The kiosk may analyze the image(s) with an algorithm (e.g., executed by the microcontroller of the kiosk) to confirm the test cartridge ID/test type. This analysis may be used to confirm the cartridge's test type (Influenza A+B, Covid-19, multiplex Influenza A+B and Covid-19, etc.). The test cartridge ID number may match the test kit ID number (also known as self-testing kit ID number), in some embodiments.

The kiosk may send the test strip result electronically to a HIPAA-compliant cloud database along with the test cartridge ID/test kit ID/test type/test order number. AES-128or AES-256 encryption may be use during the data transfer to help ensure HIPAA-compliance. The cloud database system may link the test cartridge ID, test kit ID, test type, and test strip results (and in some embodiments, the test order number as well) from the kiosk with the test order number and patient ID previously sent by the remote provider. The cloud database system may electronically send the test results to the patient, patient's physician, and/or a remote provider.

The physical kiosk may not contain identifiable patient health information to help ensure HIPAA-compliance in the event of hacking of the physical kiosk. The test results-related data (e.g., test cartridge ID, test kit ID, test type, test kit ID, test strip result, test order number) may be linked to identifiable patient health information in the HIPAA-compliant cloud rather than at the physical kiosk.

If the patient previously opted-in to sending their individual test results to their employer, the cloud database may also send the test results to their employer. The cloud database will not send the employee's individual test results to their employer if the employee did not opt-in. This opt-in method for sharing the employee's individual test results with their employer helps ensure HIPAA-compliance.

The conveyor belt may bring the test cartridge back to the robotic pipette module (e.g., at a second location in the kiosk) and/or the robotic pipette module may adjust to the location of the test cartridge. The robotic pipette module may use the disposable pipette tip to enter the wash buffer chamber/vial in the test cartridge. The robotic pipette module may use the pipette tip to aspirate the wash buffer. The robotic pipette module may dispense the wash buffer through the pipette tip into the vial with the mixed patient sample and buffer extraction solution and/or onto the test strip. The robotic pipette module may dispense the wash buffer onto any surfaces on the test cartridge that has touched the patient sample (such as the self-sealing vial cap). This process may chemically decontaminate the regulated medical waste (e.g., patient nasal sample) and converts the medical waste into standard municipal waste.

The robotic pipette module may drop the disposable pipette tip back into the original holder/holster of the disposable pipette tip in the test cartridge for eventual disposal with the entire test cartridge. The conveyor belt may drop the entire test cartridge into a waste receptacle (e.g., an internal waste bin within the kiosk) for disposal. An ultraviolet light above the waste receptacle may activate as an extra layer of safety to decontaminate any residual viruses or bacteria that may reside within the waste receptacle.

Referencing FIGS. 10-13, a self-service diagnostic kiosk will now be described. The kiosk may have a conveyor belt 2 for transporting test strips 210 (in alternative embodiments, the test strips on the conveyor belt could be test cartridges 200 instead) to the robotic pipette module 5, imaging module 9, and off the conveyor for disposal. This may include the conveyor belt transporting tests to a first location, a second location, a third location, etc. within the kiosk. The conveyor belt may also enable parallel processing of multiple test strips holding multiple patient samples simultaneously. The conveyor belt may hold, convey, and transport a singular or multiple test strip(s) to different locations in the device throughout the testing/analysis/waste disposal process, in various embodiments. The conveyor belt may also have multiple slots 14 to hold multiple test strips and allow for parallel processing.

The kiosk may have a robotic pipette module 5 for aspirating a fixed volume of the mixed buffer extraction solution and patient nasal fluid sample from the cartridge vial, and transporting and dispensing the mixed solution onto the test strip on the conveyor belt. The robotic pipette module may pick up and use the disposable pipette tip that comes with the test cartridge for the aspiration, dispensing, and transportation.

The kiosk's robotic pipette module may use a robotic motion system 6 (e.g., controlled by a microcontroller of the kiosk) that allows for cartesian linear movement (x-direction, y-direction, and/or z-direction) and rotational/polar movement (theta direction and/or phi direction). This motion system may allow the robotic pipette module to move to and access different components within the test cartridge 200 (in some embodiments, the test cartridge is on the vortex stand/holding cavity 17) and test strip(s) 210 on the conveyor belt 2 and/or transverse across different sections of the conveyor belt. In some embodiments, the robotic motion system may include a linear actuator 7 for linear motion and a servo motor 8 for rotational/polar motion.

The kiosk may also have a vortex mixer 4 that vibrates and mixes the patient sample (e.g., nasal sample, saliva) with buffer extraction solution, which release the viral antigen protein in the patient sample from the viral shell. The vibration of the vortex mixer may be controlled by a microcontroller 11 of the kiosk and/or based on a force used to engage the vortex mixer (e.g., a plunger 15 on the robotic pipette module that presses down upon the vortex mixer). The vortex mixer may directly interface with and vibrate the test cartridge's vial that holds buffer extraction solution and the patient sample (e.g., to generate a mixture). In some embodiments, the vortex mixer may include a holding cavity (also known as, vortex stand) 17 that holds the test cartridge and vial before, during, and after vortex mixing.

The kiosk may have a high-quality, imaging system (e.g., including a high-quality camera) 9 that captures images of the test strip on the conveyor belt and the test strip's test result line(s) and control line for analysis. A backend image processing algorithm (e.g., stored within a memory of the kiosk and/or executed by a microcontroller of the kiosk) may be used to determine the test result from the image of the test strip. The imaging system may be within an enclosure 10 that controls lighting and prevents non-desired background light from reaching the test strip during imaging.

The kiosk may have a holder (also known as, vortex stand or holding cavity) 17 above the vortex mixer 4 into which a user can place the test cartridge, so that the cartridge's vial holding the buffer extraction solution and patient sample can be mixed by the vortex mixer. When the test cartridge is fully loaded into this holder, the cartridge's vial may be directly touching the vortex mixer and be inside the vortex mixer's holding cavity.

Referencing now to FIG. 13 the kiosk may include one or more optointerruptors 12 used to control the positioning of the test strips underneath the imaging system. The kiosk may include optointerruptors for positioning the default positioning of the robotic pipette module (for example, near the top of linear actuator rail 7). The kiosk may include a plunger 15 attached to the robotic pipette module, which may be used to apply pressure onto the cartridge vial and the vortex mixer (FIG. 13C). This pressure can be used to directly interface and engage the vial with the vortex mixer during vortex vibration and mixing, and tightly push the vial against the vortex mixer during vortex vibration and mixing to enhance the mixing efficiency). The vortex mixer can be activated by this pressure. Alternatively, the vortex mixer can be activated through an electronic signal from the device.

Referencing FIG. 10, an example test cartridge will now be described. The test cartridge may include a holster/holder that holds a disposable pipette tip 207. The test cartridge may have a vial with buffer extraction solution 204. The user may place the patient sample (e.g., nasal fluid sample on nasal swab) into this vial. The vortex mixer may vibrate this vial to mix the buffer extraction solution with the patient sample to elute the nasal fluid sample from the nasal sample with high efficiency, and to release the viral antigen proteins from the viral shell in the patient sample. The test cartridge may include a seal-scaling cap (e.g., septum cap) 206 on top of the vial. This self-scaling cap may allow the pipette tip on the robotic pipette module to enter the vial and aspirate the mixed solution and leave. When the pipette tip leaves the vial, the cap may self-seal. The self-scaling cap may reduce or eliminate aerosolized mixed sample from escaping the vial, which may reduce cross-contamination potential. The user may open the cap before placing the sample into the vial. The user may then close the cap. The test cartridge may have a flexible o-ring 205 that holds the sample vial tightly to the cartridge, while allowing the vial to vibrate around the free space surrounding the vial during vortex mixing (FIG. 13C).

Referencing FIGS. 10 and 13, a method and a process will be described for an automated diagnostic testing process that may be performed using a self-service diagnostic kiosk 110 as described herein.

The user/patient may self-collect their sample (e.g. nasal sample) with the self-collection nasal swab 310 (FIGS. 6 and 15C). In some embodiments, the user/patient may self-collect a nasal fluid sample from their nostrils using a self-collection nasal swab.

Referencing FIGS. 10 and 13, the user/patient may place their self-collected sample in the test cartridge 200 (FIG. 10). The user/patient may open a cap on the test cartridge, place the nasal swab holding their nasal fluid sample in the vial underneath the cap, break the swab handle off of the swab tip by pulling the handle at the handle's breakpoint against the vial, and secure/close the cap back onto the vial.

After self-collecting a sample, the user/patient may load the test cartridge into the test cartridge holder on the device, above the vortex mixer (FIG. 13B). When the test cartridge is fully loaded, the cartridge's vial will be directly touching the vortex mixer and be inside the vortex mixer's holding cavity.

The user/patient may load a test strip 210 directly onto the device's conveyor belt 2 (FIG. 13B) (a first location). Optointerrupter(s) 12 may sense when the test strip has been loaded onto the conveyor belt. The user/patient may also activate the kiosk. The conveyor belt may then bring the test strip to the robotic pipette module (a location on the belt accessible by the robotic pipette module; in some embodiments, the second location) (FIGS. 13C and 13F). The motion system 6 may lower the robotic pipette module 5 via the linear actuator 7 until the plunger 15 of the robotic pipette module reaches the vial 204 of the test cartridge 200. The robotic pipette module 5 may use a plunger 15 to push against the cartridge's vial 204 and vortex mixer 4 to activate the vortex mixing and mix the buffer extraction solution with the nasal fluid sample inside the vial (FIG. 13C). The nasal fluid may be eluted from the nasal swab into the buffer extraction solution. The nasal fluid may be mixed with the buffer extraction solution and the viral antigen proteins in the patient sample may be released from the viral shell into the mixed solution.

The linear actuator may raise the robotic pipette module, and the servo motor 8 may rotate the robotic pipette module (in some embodiments, clockwise) to bring it above the pipette tip 207 on the test cartridge. The linear actuator may lower the robotic pipette module to pick up the disposable pipette tip 207 from the test cartridge (FIG. 13D). The linear actuator may raise the pipette module, and the servo motor may rotate the pipette module (in some embodiments, counter-clockwise) to position the pipette module above the cap 206 and vial 204 of the test cartridge. The linear actuator may lower the robotic pipette module, so that it may enter the vial with the disposable pipette tip. The robotic pipette module may aspirate a fixed volume of mixed solution/sample into the pipette tip. The linear actuator may raise, so that the robotic pipette module/pipette tip may leave the vial, and the cap may self-seal. The servo motor may rotate (in some embodiments, clockwise) to move and bring the robotic pipette module with the pipette tip over the test strip on the conveyor belt (in some embodiments, the second location) (FIG. 13F). Then, the pipette module may dispense the mixed solution/sample from the pipette tip onto the test strip and into the test strip's sample well (FIG. 13F). The linear actuator may raise the pipette module, the servo motor may rotate the pipette module (in some embodiments, counterclockwise), and the linear actuator may lower the pipette module to position the pipette module above the holster and original location of the pipette tip on the test cartridge. The pipette module ejects the pipette tip, which drops back into its holster on the test cartridge in preparation for disposal with the test cartridge (FIG. 13G). The linear actuator raises the pipette module back to its original default position. Optointerrupter(s) may sense when the pipette module has returned to its original position. The microcontroller 11 may control the entire positioning and aspiration and dispense process of the conveyor belt 2 and the robotic pipette module 5 and its motion control system 6.

The test strip may then develop over a pre-specified period of time (e.g., 10-15 minutes) that may be dependent on the test strip type within the test cartridge (Influenza A+B (e.g., 10 minutes), Covid-19 (e.g., 15 minutes), multiplex Influenza A+B/Covid-19 (e.g., 15 minutes), etc.) (FIG. 13H). When sufficient time has passed, the conveyor belt may bring the test strip underneath the imaging system (in some embodiments, a third location). Optointerrupters 12 may be used to sense and determine when the test strip has been correctly positioned underneath the imaging module. Alternatively, the imaging module may use a continuous video feed and a positioning algorithm that analyzes the video feed to correctly position the test strip underneath the imaging module. After the test strip has been correctly positioned underneath the imaging module, the imaging system may capture an image of the test strip for analysis of the test result (FIG. 13I) The kiosk may then analyze the test strip image for the test result with an image processing algorithm (e.g., executed by a microcontroller of the kiosk) to determine the test strip result (e.g., the presence or absence of one or more patient conditions). The image processing algorithm may be a deterministic geometric image processing algorithm.

The conveyor belt may drop the test strip off of the conveyor belt for disposal. The user/patient may remove the test cartridge from the holder with their hands for disposal.

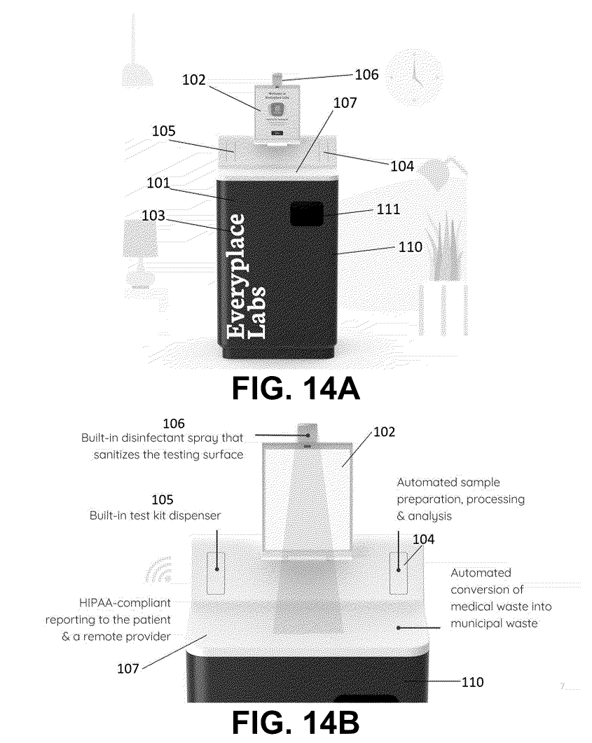

Referencing now to FIGS. 14 and 15, a self-service diagnostic kiosk will now be described. In some embodiments, the kiosk may include an integrated design that enables the kiosk to be a standalone unit (i.e., the kiosk may not need additional furniture for the patient to self-collect their sample or additional support from a medical office assistant for a patient to pick up a self-testing kit). The kiosk may be welcoming and reduce or eliminate cross-contamination areas.

Referencing FIG. 14, the kiosk 110 may include a device shell 101 (e.g., an external device shell) that is coated with antimicrobial coating 103. All or part of the external surfaces of the kiosk and/or the kiosk shell may be coated with antimicrobial coating. The kiosk may include a work surface (also known, as a self-testing surface) 107 where the patient can self-collect their sample (e.g., nasal swab sample). The work surface may be coated with antimicrobial coating. The kiosk may include an external waste receptacle 111 (e.g., waste bin) that is externally accessible by the patient to discard municipal waste/non-regulated medical waste, such as wrappers and packaging that originally contained the test cartridge and nasal swab. The kiosk may also include a port 105 configured to dispense self-testing kits (and in some embodiments, medication as well) to the patient. The self-testing kits may contain test cartridges 200 and self-collection tools such as self-collection nasal swabs 310. The self-testing kits may also contain sanitization wipes that the patient/user can use to wipe the work surface and kiosk to feel more peace of mind that the work surface and kiosk is clean. The patient/user can also use these hand sanitization wipes to clean their hands before self-collecting their sample. The kiosk may store a repository of test cartridges or a repository of a plurality of test cartridges for different test types (in some embodiments, below or nearby the port 105). Because the kiosk may include a display 102 for guiding the patient through the entire self-collection and self-testing process, instructions may not be included in the self-testing kit, which may result in a smaller self-testing kit. The self-testing kit may include a small box or small wrapper holding the test cartridge and self-collection tool (e.g., nasal swab). The test cartridge and self-collection tool (e.g., nasal swab) may be contained within wrappers as well. The kiosk may include a port 104 for the patient to insert the test cartridge holding the patient sample into the kiosk for analysis. The kiosk may include a display 102 (e.g., a LED display, a LCD, a cathode ray tube, etc.) configured to guide the patient through the entire sample self-collection process with the self-testing kit and the self-testing process with the kiosk. The display may guide the patient through how to use the self-testing kit, test cartridge, self-collection tool, and the kiosk. The bottom of the display may include a scanner for scanning the test order number on the patient's phone during the patient check-in process at the kiosk. The kiosk may include a cleaning module 106 with a spraying mechanism for disinfectant spray and/or ultraviolet light to sanitize the testing surface and the kiosk between patients. In some embodiments, the cleaning module may include a reel-to-reel film (e.g., similar to the film on a patient bed at a physician consultation room) over the work surface that could be automatically swapped out between patients. Additionally or alternatively, the cleaning module may include a windshield wiping mechanism used to fully wipe and clean the work surface of the kiosk. Further, in some embodiments, the work surface may have an anti-microbial coating applied thereto to reduce contamination. Such mechanisms for cleaning the kiosk may be engaged by a microcontroller of the kiosk between patients such that the kiosk cleans itself and/or may be used by a patient, technician, janitorial staff, or other staff to clean the kiosk.

Referencing FIG. 15C, a test cartridge 200 will now be described. The test cartridge may include a pivoting cover that the patient can pivot open to insert the sample and pivot closed after inserting the sample. The test cartridge may also include a contoured design that enables directional loading of the test cartridge into the kiosk, which may reduce or eliminate user errors.

Referencing FIG. 15, a method and workflow for the patient interacting with the kiosk will now be discussed.

A patient (in some embodiments, an employee of a worksite) experiences symptoms of illness, such as respiratory symptoms similar to Influenza and Covid-19. The patient may see a remote provider over a telehealth platform, and the remote provider on a telehealth platform may order a test for the patient/user, and send the test order number/reservation number to the patient/user and the patient's phone. Alternatively, instead of consulting with a remote provider on a telehealth platform, a patient can fill out an online screening and triage tool on their phone, tablet, or laptop. Based upon the patient's symptoms (such as respiratory symptoms), an online system or the online screening and triage tool may order a lab test for the patient and send the test order number/reservation number to the patient and the patient's phone, along with instructions that guides the patient to a kiosk location. The kiosk may be at a private office room or private wellness room at a worksite, such as a manufacturing line. The patient/user may “check-in” at the kiosk by scanning a test order number/reservation number on their phone on the kiosk's scanner (FIG. 15A). The numbers may be embedded as a QR code or barcode on the patient's phone that can be scanned by the kiosk. Alternatively, NFC can also be used.

Based upon the test order number/reservation number, the kiosk may dispense the appropriate self-testing kit(s) 301 to the patient through the self-testing kit dispensing port 105 (FIG. 15B).

The patient may then open the self-testing kit 301, which contains a test cartridge 200 (FIG. 15C), a self-collection tool (e.g., nasal swab) 310 (FIG. 15C), and sanitization wipes. The self-testing kit may be in a relatively small box, sleeve, wrapper, or blister pack format.

The patient may sanitize their hands and the testing surface with the sanitization wipes.

The patient may collect their sample using the self-collection tool (e.g., nasal swab) (FIG. 15C) and insert sample into the test cartridge. In some embodiments, the patient may insert a swab with a nasal sample into the test cartridge (FIG. 15C).

The patient may insert/deposit the test cartridge into the kiosk through the test cartridge insertion port 104 (FIG. 15D).

The patient may throw away any trash from the self-testing kit into the external waste receptacle 111 of the kiosk (FIG. 14A). In some embodiments, the patient may leave the kiosk and wait for their results elsewhere.

The kiosk may automate the sample preparation (mixing), processing, and analysis of the sample in the test cartridge with processes and mechanisms described in other embodiments of the kiosk. The kiosk may convert the regulated medical waste in the test cartridge into standard municipal waste and discard the test cartridge into an internal waste container inside of the kiosk, using processes and mechanisms described herein. The results may be produced within 5-15 minutes, depending on the test (FIG. 15E). The results may be electronically sent to the patient's phone, a physician of the patient, and/or a remote provider.

The kiosk may sanitize the testing surface and the kiosk through a sanitization spray and/or ultraviolet light or other mechanisms (FIG. 14B). The kiosk may initiate the sanitization process after the patient leaves the kiosk. The kiosk may not need to wait for the test result to be ready before initiating the sanitization process. After the sanitization process is complete, the next patient may use the kiosk (including inserting a test cartridge into the kiosk), so the kiosk may parallel process the analysis of multiple test cartridges from multiple patients simultaneously, using mechanisms and processes described in other embodiments. Alternatively, the kiosk may wait for the test results to be ready and for the patient to leave the kiosk before initiating the sanitization process. Motion sensing by the kiosk may be used to sense when the patient has left the kiosk.

The kiosk display 102 (FIGS. 14 and 15) may guide the patient throughout this method and workflow and the entire sample self-collection and self-testing process with the self-testing kit and the kiosk. The patient can navigate through kiosk display instructions with voice activation or motion sensing by the kiosk, enabling a contactless experience. Additionally and alternatively, through sensors (such as optointerrupters) at various locations on the kiosk (such as at the test cartridge insertion port), the kiosk may detect when the patient has completed certain steps and actions, triggering the next instruction screen to be shown on the display. Alternatively, the patient can navigate the instructions through a touchscreen capability on the display. Further, the display may be coated with antimicrobial coating to ensure cleanliness, safety, and prevent cross-contamination and spread of disease.

A method for the self-service diagnostic kiosk to process, analyze, and test venous, whole blood samples that are self-collected by patients will now be discussed. The patient may self-collect a venous, whole blood sample using an existing self-collection method/tool for self-collecting venous, whole blood. The patient may then place the venous, whole blood sample into a test cartridge and insert the test cartridge into the self-service diagnostic kiosk. The kiosk may contain a centrifuge that spins the self-collected venous, whole blood sample in the test cartridge and separates the plasma/serum in the whole blood from the whole blood and blood cells (e.g., red blood cells, white blood cells, etc.). The kiosk may auto-adjust a counterweight in the centrifuge (e.g., based on a command signal provided by a microcontroller of the kiosk) based upon the weight/volume of the sample to match the weight/volume of the sample. The counterweight can be a fluid container holding a fluid such as water, and the kiosk would auto-adjust the volume of the fluid in the container, so that its weight/volume reflects and matches the weight/volume of the blood sample. The kiosk may dispense the separated plasma/serum sample onto the test strip for analysis. By automatically separating the plasma/serum from whole blood, the kiosk is able to run a wider range of blood tests from blood that is self-collected by the patient.

The self-service diagnostic kiosk may also aggregate population health data and trends and/or monitor the incidence of respiratory diseases, infectious diseases, and other diseases in the workplace and other community settings where the kiosk is deployed. The self-service diagnostic kiosk may also generate population health reports with the data and trends and notify/warn the employer and other stakeholders that manage the workplace and other community settings where the kiosk is deployed that the incidence of respiratory diseases, infectious diseases, and other diseases is increasing, allowing the stakeholders to take action to prevent potential outbreaks of disease in the community settings.

The self-service diagnostic kiosk may be an Internet of Things-enabled and internet connected device (via Wi-Fi, cellular networks, or other mediums and networks), and may integrate with the electronic health record of an employer health or telehealth provider. The self-service diagnostic kiosk may provide automated and proactive test replenishment, maintenance, and quality assurance via internet of things (IoT) and the kiosk's connectivity capabilities. The self-service diagnostic kiosk may monitor the supply and usage of test kits, so that the supply of test kits can be automatically and proactively replenished as test kits are used and/or before test kits run out. The self-service diagnostic kiosk may use internal signals to monitor the internal health of the self-service diagnostic kiosk and determine if parts/sub-systems/the entire kiosk needs maintenance, decommissioning, or replacement and/or to predict if parts/sub-systems/the entire kiosk will need maintenance, decommissioning, or replacement in the future. The self-service diagnostic kiosk may be modular to enable maintenance and replacement of the parts/sub-systems/the entire kiosk. The modularity may also enable easy self-maintenance and self-replacement of parts/sub-systems/the entire kiosk by the stakeholders who manage the community settings where the kiosk is deployed. The kiosk may predict when new modules need to be shipped to the stakeholders before the existing modules/parts/sub-systems/the entire kiosk breaks and needs to be replaced. The self-service diagnostic kiosk may dispense quality assurance kits with quality assurance cartridges periodically, aperiodically, and/or when quality assurance is needed or scheduled. The self-service diagnostic kiosk may dispense test kits that contain a dual test cartridge-quality assurance cartridge periodically, aperiodically, and/or when quality assurance is needed or scheduled. The self-service diagnostic kiosk may dispense test kits that contain a test cartridge and a quality assurance cartridge periodically, aperiodically, and/or when quality assurance is needed or scheduled.

The self-service diagnostic kiosk may include ultraviolet light(s) that illuminate parts of or all of the insides/internals of the kiosk to decontaminate the insides/internals of the kiosk between patient usage. Example areas where the ultraviolet light(s) may illuminate include the test cartridge insertion port, the vortex mixer, the conveyor belt that transports the test cartridge to different locations within the kiosk during the analysis process, the robotic pipette module, the imaging system, and/or air spaces throughout the insides of the kiosk.

The self-service diagnostic kiosk can perform a wide range of rapid tests including, but not limited to, Influenza A+B, Covid-19, multiplex Influenza A+B and Covid-19, drug tests, general urinalysis, chronic diseases, etc. The test cartridges may be customized to the test type by having test strip(s) inside that are custom to the test type. As an example, a test cartridge that can perform a multiplex Influenza A+B and Covid-19 test can contain a multiplex antigen-based, lateral flow immunoassay, test strip for Influenza A+B and Covid-19. A test cartridge that can perform an Influenza A+B test can contain an antigen-based, lateral flow immunoassay test strip for Influenza A+B. A test cartridge that can perform a Covid-19 test can contain an antigen-based, lateral flow immunoassay test strip for Covid-19.

The self-service diagnostic kiosk may track when certain actions have been completed by the user/patient with sensors. The kiosk (e.g., a microcontroller of the kiosk) may use these sensed inputs to determine when the kiosk should perform certain actions/change states/activate mechanisms/change the instructions page on the display, and also to change the instructions page on the display to the next instructions page on the screen (or to the prior instructions page). As an example, when the user/patient scans their phone on the kiosk to “check-in” to the kiosk, the kiosk may sense this input, automatically dispense the appropriate test kit, and change the instruction page on the display to go to an instruction page that guides the user/patient on how to use the test kit and collect their sample. As another example, when the user/patient inserts the test kit into the test cartridge insertion port, the kiosk may sense this input, automatically activate test cartridge processing/analysis mechanisms to analyze the test cartridge, and change the instructions page on the display to let the patient know they can leave the kiosk.

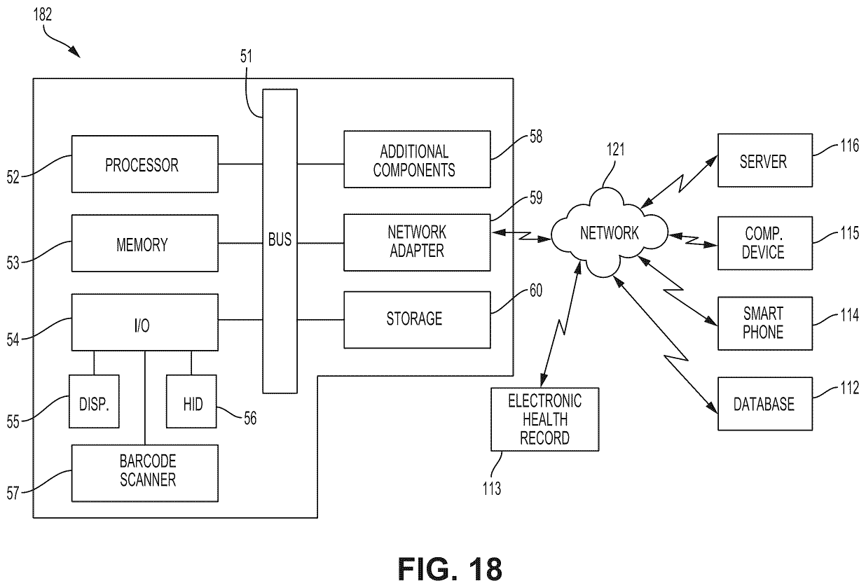

As indicated herein, the self-diagnostic kiosk may include one or more computing devices. For example, the self-diagnostic kiosk may include a microcontroller (e.g., a general-purpose processor and/or an application-specific integrated circuit (ASIC)). The microcontroller may include and/or be communicatively coupled to a memory, which may include instructions that are executable by the microcontroller (e.g., by a processor of the microcontroller) to perform one or more functions. For example, the microcontroller may execute the instructions to operate a vortex mixer of the self-diagnostic kiosk (e.g., by causing the vortex mixer to vibrate directly, or by causing a plunger of a robotic pipette module to engage the vortex mixer thereby causing the vortex mixer to vibrate), operate a conveyor belt of the self-diagnostic kiosk, operate a robotic pipette module of the self-diagnostic kiosk, operate an imaging system of the self-diagnostic kiosk, receive an image of the test strip from an imaging system of the self-diagnostic kiosk, analyze the image of the test strip to determine whether one or more patient conditions is present, operate a display of the self-diagnostic kiosk, and/or communicate with one or more computing devices external to the self-diagnostic kiosk (e.g., to transmit results of the self-diagnostic test). An example microcontroller (e.g., a sample computing device) will now be shown and described with reference to FIG. 18.

Referring now to FIG. 18, an illustrative computing device 182 (e.g., microcontroller) will now be discussed in greater detail, without limitation. The computing device 182 may include a processor 52, memory 53, network controller 59, and optionally an input/output (I/O) interface 54. Skilled artisans will appreciate additional embodiments of a computing device that may omit one or more of the components or include additional components without limitation. The processor 52 may receive and analyze data. The memory 53 may store data, which may be used by the processor 52 to perform the analysis. The memory 53 may also receive data indicative of results from the analysis of data by the processor 52.

The memory 53 may include volatile memory modules, such as random access memory (RAM), and/or non-volatile memory modules, such as flash based memory. Skilled artisans will appreciate the memory to additionally include storage devices, such as, for example, mechanical hard drives, solid state data, and removable storage devices.

The computing device may also include a network controller 59. The network controller 59 may receive data from other components of the computing device to be communicated with other computing devices 112, 113, 114, 115, 116 via a network 121. The communication of data may be performed wirelessly. More specifically, without limitation, the network controller 59 may communicate and relay information from one or more components of the computing device, or other devices and/or components connected to the computing device, to additional connected devices. Connected devices and/or software are intended to include databases 112, computer 115, mobile computing devices, smartphones 114, tablet computers, electronic health records 113, data servers 116, and other electronic devices that may communicate digitally with another device. In one example, the computing device may be used as a server to analyze and communicate data between connected devices.

The computing device 182 may also include an I/O interface 54. The I/O interface 54 may be used to transmit data between the computing device and extended devices. Examples of extended devices may include, but should not be limited to, a display, external storage device, human interface device, printer, sound controller, barcode scanner, or other components that would be apparent to a person of skill in the art. For example, the I/O interface 54 may be used to with a barcode and/or RFID scanner 57 to detect an identification of a patient and electronically communicate such identifying information, for example, via WIFI, BLUETOOTH, and/or another network. Additionally, one or more of the components of the computing device may be communicatively connected to the other components via the I/O interface 54.

The components of the computing device 182 may interact with one another via a bus 51. Those of skill in the art will appreciate various forms of a bus that may be used to transmit data between one or more components of an electronic device, which are intended to be included within the scope of this disclosure.

The computing device 182 may communicate with one or more connected devices via a network 121. The computing device 182 may communicate over the network 121 by using its network controller 59. More specifically, the network controller 59 of the computing device may communicate with the network controllers of the connected devices 114, 115, databases 112, and electronic health records 113. The network 121 may be, for example, the internet. As another example, the network 121 may be a WLAN. However, skilled artisans will appreciate additional networks to be included within the scope of this disclosure, such as intranets, local area networks, wide area networks, peer-to-peer networks, BLUETOOTH, RFID, and various other network formats. Additionally, the computing device and/or connected devices may communicate over the network via a wired, wireless, or other connection, without limitation.

Referencing FIGS. 8, 11, and 12, the imaging module 9 and the robotic pipette module 5 may connect to the serial interface of the microcontroller 11. The microcontroller may control these devices through the use of publicly available software libraries. The LEDs of the imaging module and vortex mixer 4 may connect to the digital outputs of the microcontroller. To enable these devices, the microcontroller outputs a digital signal for a period of time. Sensors used for object detection, such as optointerruptors 12, proximity sensors, and limit switches, may connect to the digital inputs of the microcontroller. When the sensor detects the object, it may output a digital signal, which is read by the microcontroller. Stepper motors 13, 16 that control the movement of the conveyor belt 2 and the linear actuator 7, respectively, may be driven by stepper motor drivers. These drivers may interface with the digital outputs of the microcontroller and translate the simple low voltage direction and step signals from the microcontroller to higher voltage signals for precision control of the stepper motor. The servo motor 8 may connect to the pulse-width modulation (PWM) interface on the microcontroller. In order to control the stepper motor's position, the microcontroller may modulate its frequency. A specific frequency on the output signal may correspond to a specific position on the stepper motor.

As described above, in some embodiments the microcontroller may communicate with one or more external computing devices. For example, at least part of the data detected and/or analyzed during operation may be communicated with an electronic health record 113. For example, data and results from analysis of the data may be communicated to a collection of patient information on an electronically-stored medium. The electronic health record 113 may include additional data relating to a patient, some of which may have been communicated to the electronic health record 113 from other medical professionals and/or procedures. Analysis detected from the sample received by the patient may be compared to data present in the electronic health record 113 to detect a likelihood of a health risk or to perform other advanced calculations. This additional data may include, but should not be limited to, demographics, medication allergies, immunizations received, medical history, prior laboratory tests and corresponding results, vital signs, radiology charts, age, weight, body mass index (BMI), blood tests, and other medical information. The electronic health record 113 may additionally include health insurance policy, billing details, and other information related to the administration of medical services.

In this embodiment, the data will be transferred using an encrypted methodology that is in compliance with HIPAA. Compliance with HIPAA is critical to be used when connecting to an electronic health record. Examples of encryption techniques that are covered can include AES 256-bit encryption, SHA-256 hashing, etc. In order to maintain this encryption standards, the data processing component may be patched using a wired or wireless connection, with data that is transmitted using Secure Socket Layers (SSL), Transport Layer Security (TLS), or the latest industry standards.

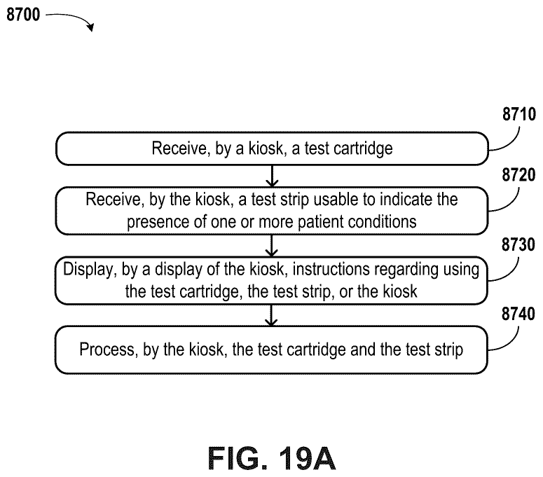

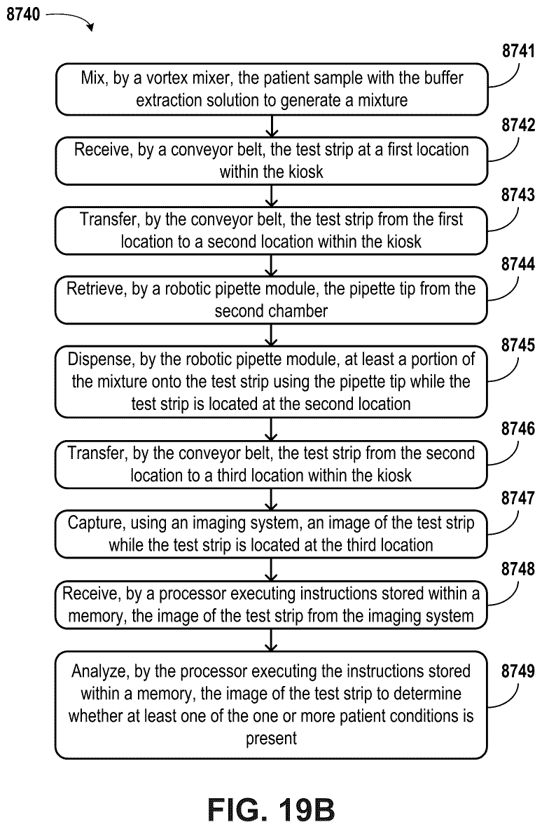

FIGS. 19A and 19B are flowchart illustrations of methods, according to example embodiments. The methods described may include one or more operations, functions, or actions as illustrated by one or more of the illustrated blocks. Although the blocks are illustrated in a sequential order, these blocks may in some instances be performed in parallel, or in a different order than those described herein. Also, the various blocks may be combined into fewer blocks, divided into additional blocks, or removed based upon the desired implementation. Further, additional blocks describing additional, non-essential steps may be included in some variations of the methods contemplated herein.

FIG. 19A is a flowchart illustration of a method 8700, according to example embodiments. The method 8700 may be performed by a self-service diagnostic kiosk as described herein.

At block 8710, the method 8700 may include receiving, by a kiosk, a test cartridge. The test cartridge may include a first chamber configured to store a patient sample and a buffer extraction solution. The test cartridge may also include a second chamber configured to store a pipette tip.

At block 8720, the method 8700 may include receiving, by the kiosk, a test strip usable to indicate the presence of one or more patient conditions.

At block 8730, the method 8700 may include displaying, by a display of the kiosk, instructions regarding using the test cartridge, the test strip, or the kiosk.

At block 8740, the method 8700 may include processing, by the kiosk, the test cartridge and the test strip.

FIG. 19B is a flowchart illustration of a method. The method illustrated in FIG. 19B may correspond to block 8740 of FIG. 19A. In other words, processing, by the kiosk, the test cartridge and the test strip in block 8740 of the method 8700 may include the blocks illustrated and described with respect to FIG. 19B.

Block 8741 may include mixing, by a vortex mixer, the patient sample with the buffer extraction solution to generate a mixture.

Block 8742 may include receiving, by a conveyor belt, the test strip at a first location within the kiosk.

Block 8743 may include transferring, by the conveyor belt, the test strip from the first location to a second location within the kiosk.

Block 8744 may include retrieving, by a robotic pipette module, the pipette tip from the second chamber.

Block 8745 may include dispensing, by the robotic pipette module, at least a portion of the mixture onto the test strip using the pipette tip while the test strip is located at the second location.

Block 8746 may include transferring, by the conveyor belt, the test strip from the second location to a third location within the kiosk.

Block 8747 may include capturing, using an imaging system, an image of the test strip while the test strip is located at the third location.

Block 8748 may include receiving, by a processor executing instructions stored with a memory, the image of the test strip from the imaging system.

Block 8749 may include analyzing, by the processor executing the instructions stored within a memory, the image of the test strip to determine whether at least one of the one or more patient conditions is present.

### II. CONCLUSION

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all of the message flow diagrams, scenarios, and flow charts in the figures and as discussed herein, each step, block, operation, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

A step, block, or operation that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical operations or actions in the method or technique. The program code and/or related data can be stored on any type of computer-readable medium such as a storage device including RAM, a disk drive, a solid state drive, or another storage medium.

The computer-readable medium can also include non-transitory computer-readable media such as computer-readable media that store data for short periods of time like register memory and processor cache. The computer-readable media can further include non-transitory computer-readable media that store program code and/or data for longer periods of time. Thus, the computer-readable media may include secondary or persistent long term storage, like ROM, optical or magnetic disks, solid state drives, compact-disc read-only memory (CD-ROM), for example. The computer-readable media can also be any other volatile or non-volatile storage systems. A computer-readable medium can be considered a computer-readable storage medium, for example, or a tangible storage device.

Moreover, a step, block, or operation that represents one or more information transmissions can correspond to information transmissions between software and/or hardware modules in the same physical device. However, other information transmissions can be between software modules and/or hardware modules in different physical devices.

The particular arrangements shown in the figures should not be viewed as limiting. It should be understood that other embodiments can include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims.

Embodiments of the present disclosure may thus relate to one of the enumerated example embodiments (EEEs) listed below.

EEE 1 is a system comprising:

a test cartridge comprising:
a first chamber configured to store a patient sample and a buffer extraction solution; and
a second chamber configured to store a pipette tip;

a test strip usable to indicate the presence of one or more patient conditions; and

a kiosk configured to receive and process the test cartridge and the test strip, wherein the kiosk comprises:

a vortex mixer configured to mix the patient sample with the buffer extraction solution to generate a mixture;

a conveyor belt configured to:
receive the test strip at a first location within the kiosk;
transfer the test strip from the first location to a second location within the kiosk; and
transfer the test strip from the second location to a third location within the kiosk;

a robotic pipette module configured to retrieve the pipette tip from the second chamber and dispense at least a portion of the mixture onto the test strip using the pipette tip while the test strip is located at the second location;

an imaging system configured to capture an image of the test strip while the test strip is located at the third location;

a display configured to display instructions regarding using the test cartridge, the test strip, or the kiosk; and

a processor communicatively coupled to the vortex mixer, the conveyor belt, the robotic pipette module, the imaging system, and the display, wherein the processor is configured to execute instructions stored within a memory to:
operate the vortex mixer;
operate the conveyor belt;
operate the robotic pipette module;
operate the imaging system;
receive the image of the test strip from the imaging system;
analyze the image of the test strip to determine whether at least one of the one or more patient conditions is present; and
operate the display.

EEE 2 is the system of EEE 1, wherein the test cartridge comprises an ergonomic shell, wherein the ergonomic shell is made of moisture-impermeable material, and wherein the moisture-impermeable material comprises a seal layer and a desiccant.

EEE 3 is the system of EEE 2, wherein the robotic pipette module is configured to pierce the seal layer in order to access one or more components of the test cartridge.

EEE 4 is the system of either EEE 2 or EEE 3, wherein the test strip is configured to be stored within the test cartridge, and wherein the seal layer is configured to encapsulate the test strip.

EEE 5 is the system of any of EEEs 1-4, wherein test cartridge comprises an external label, wherein the external label comprises a barcode indicative of a test cartridge number associated with the test cartridge, a test order number, and a test kit identification number, and wherein the test cartridge number is usable to link results of the image analysis performed by the processor to a patient associated with the patient sample.

EEE 6 is the system of EEE 5,

wherein:
(i) the image of the test strip includes a portion corresponding to the external label; and
(ii) the processor is further configured to analyze the image of the test strip to determine the test cartridge number associated with the test cartridge, or

wherein:
(i) the imaging system is further configured to capture an image of the external label; and
(ii) the processor is further configured to:
   receive the image of the external label from the imaging system; and
   analyze the image of the external label to determine the test cartridge number associated with the test cartridge.

EEE 7 is the system of any of EEEs 1-6, wherein, prior to the kiosk receiving and processing the test cartridge, the test cartridge is configured to be stored in a self-collection kit.

EEE 8 is the system of EEE 7, wherein the self-collection kit comprises:

a box having a kit label, wherein the kit label comprises a barcode usable to link contents of the self-collection kit with an analysis performed using the kiosk;

a peelable layer configured to encapsulate, prior to the kiosk receiving and processing the test cartridge:
the test cartridge; and
a self-collection tool usable by a patient to self-collect the patient sample; and

one or more sanitization wipes disposed on the peelable layer.

EEE 9 is the system of any of EEEs 1-8, wherein the test strip is configured to be stored within the test cartridge, and wherein:

the conveyor belt receiving the test strip at the first location comprises receiving the test cartridge at the first location;

the conveyor belt transferring the test strip from the first location to the second location comprises transferring the test cartridge from the first location to the second location; and

the conveyor belt transferring the test strip from the second location to the third location comprises transferring the test cartridge from the second location to the third location.

EEE 10 is the system of EEE 9, wherein the conveyor belt is further configured to, upon the imaging system capturing the image of the test strip while the test strip is located at the third location, transfer the test cartridge from the third location to the second location, and wherein the robotic pipette module is further configured to:

retrieve a wash buffer using the pipette tip; and

dispense the wash buffer into first chamber using the pipette tip.

EEE 11 is the system of EEE 10, wherein the test cartridge further comprises a third chamber configured to store the wash buffer, and wherein retrieving the wash buffer using the pipette tip comprises retrieving the wash buffer from the third chamber using the pipette tip.

EEE 12 is the system of either EEE 10 or EEE 11, wherein the wash buffer comprises diluted bleach.

EEE 13 is the system of any of EEEs 1-12, wherein the first chamber comprises a vial configured to store the patient sample and the buffer extraction solution, and wherein the vial comprises a self-sealing cap.

EEE 14 is the system of EEE 13, wherein the test cartridge further comprises a flexible ring configured to:

retain the vial within the first chamber; and

allow the vial to be rotated by the vortex mixer.

EEE 15 is the system of any of EEEs 1-14, wherein the test strip comprises a lateral flow immunoassay test strip, a lateral flow aptamer assay test strip, or a clinical chemistry test strip.

EEE 16 is the system of any of EEEs 1-15, wherein the kiosk further comprises a shell treated with an antimicrobial coating layer.

EEE 17 is the system of EEE 16, wherein the kiosk further comprises a port defined within the shell through which the test cartridge may be received by the kiosk.

EEE 18 is the system of any of EEEs 1-17, wherein the kiosk further comprises one or more input conveyors configured to deliver the test cartridge to the vortex mixer or the conveyor belt.

EEE 19 is the system of any of EEEs 1-18, wherein the conveyor belt comprises a plurality of slots, and wherein each slot of the plurality of slots is configured to hold a test strip.

EEE 20 is the system of any of EEEs 1-19, wherein the kiosk further comprises a waste receptacle configured to store the test strip or the test cartridge after the imaging system has captured the image of the test strip.

EEE 21 is the system of any of EEEs 1-20, wherein the robotic pipette module comprises:

one or more linear actuators configured to translate along one or more cartesian coordinate directions; and

one or more rotational actuators configured to rotate about one or more rotational axes.

EEE 22 is the system of any of EEEs 1-21, wherein the imaging system comprises:

one or more light-emitting diodes (LEDs); and

one or more cameras.

EEE 23 is the system of EEE 22, wherein at least one of the one or more LEDs emits light having an ultraviolet wavelength.

EEE 24 is the system of any of EEEs 1-23, wherein the kiosk further comprises a communication interface configured to communicate with one or more external computing devices over a network.

EEE 25 is the system of EEE 24, wherein the one or more external computing devices comprise a cloud-based storage unit, and wherein communicating with the one or more external computing devices comprises providing, to the cloud-based storage unit:

results of the image analysis performed by the processor;

a test cartridge number associated with the test cartridge;

a test order number; or

a self-testing kit identification number.

EEE 26 is the system of either EEE 24 or EEE 25, wherein the kiosk communicates with the one or more external computing devices over the network according to a protocol that complies with one or more privacy regulations.

EEE 27 is the system of any of EEEs 24-26, wherein the one or more computing devices comprises a computing device of a patient associated with the patient sample or a computing device associated with a physician of the patient associated with the patient sample.

EEE 28 is the system of any of EEEs 24-27, wherein the network comprises the public Internet.

EEE 29 is the system of any of EEEs 1-28, wherein the kiosk further comprises one or more optointerruptors usable by the processor to:

operate the conveyor belt to:
transfer the test strip from the first location to the second location; and
transfer the test strip from the second location to the third location; or

operate the robotic pipette module to:
retrieve the pipette tip from the second chamber; and
dispense at least the portion of the mixture onto the test strip using the pipette tip.

EEE 30 is the system of any of EEEs 1-29, wherein the robotic pipette module comprises a plunger configured to engage the vortex mixer to cause the vortex mixer to mix the patient sample with the buffer extraction solution to generate the mixture.

EEE 31 is the system of any of EEEs 1-30, wherein the kiosk further comprises a work surface on which one or more items may be placed when a patient interacts with the kiosk.

EEE 32 is the system of EEE 31, wherein the kiosk further comprises a disinfectant cleaning device configured to disinfect the work surface or an external ultraviolet light source configured to disinfect the work surface.

EEE 33 is the system of EEE 32, wherein the display is further configured to display instructions that prompt the patient interacting with the kiosk to manually sanitize the work surface.

EEE 34 is the system of any of EEEs 31-33, wherein the kiosk is configured to:

store, within a repository of the kiosk, unused test cartridges, test strips in plastic wrappers, self-collection kits, self-collection tools, or medication; and

dispense an unused test cartridge, test strip, self-collection kit, self-collection tool, or medication from the repository onto the work surface in response to a request from the patient or a test order issued to the patient by the kiosk based on a request from the patient.

EEE 35 is the system of EEE 34, wherein the request from the patient is received from a mobile computing device associated with the patient.

EEE 36 is the system of either EEE 34 or EEE 35, wherein the unused test cartridges or test strips stored within the repository of the kiosk comprise a plurality of types of test cartridges or test strips usable to test for a plurality of different patient conditions.

EEE 37 is the system of any of EEEs 1-36, wherein the kiosk further comprises an externally accessible waste receptacle.

EEE 38 is a kiosk configured to receive and process a test cartridge and a test strip, wherein the kiosk comprises:

a vortex mixer configured to mix a patient sample with a buffer extraction solution to generate a mixture, wherein the test cartridge comprises:
a first chamber configured to store the patient sample and the buffer extraction solution; and
a second chamber configured to store a pipette tip;

a conveyor belt configured to:
the test strip at a first location within the kiosk, wherein the test strip is usable to indicate the presence of one or more patient conditions;
transfer the test strip from the first location to a second location within the kiosk; and
transfer the test strip from the second location to a third location within the kiosk;

a robotic pipette module configured to retrieve the pipette tip from the second chamber and dispense at least a portion of the mixture onto the test strip using the pipette tip while the test strip is located at the second location;

an imaging system configured to capture an image of the test strip while the test strip is located at the third location;

a display configured to display instructions regarding using the test cartridge, the test strip, or the kiosk; and

a processor communicatively coupled to the vortex mixer, the conveyor belt, the robotic pipette module, the imaging system, and the display, wherein the processor is configured to execute instructions stored within a memory to:
operate the vortex mixer;
operate the conveyor belt;
operate the robotic pipette module;
operate the imaging system;
receive the image of the test strip from the imaging system;
analyze the image of the test strip to determine whether at least one of the one or more patient conditions is present; and
operate the display.

EEE 39 is a test cartridge comprising:

a first chamber configured to store a patient sample and a buffer extraction solution; and

a second chamber configured to store a pipette tip,

wherein the test cartridge is configured to be received and processed by a kiosk along with a test strip usable to indicate the presence of one or more patient conditions, and

wherein the kiosk comprises:
a vortex mixer configured to mix the patient sample with the buffer extraction solution to generate a mixture;
a conveyor belt configured to:
   receive the test strip at a first location within the kiosk;
   transfer the test strip from the first location to a second location within the kiosk; and
   transfer the test strip from the second location to a third location within the kiosk;
a robotic pipette module configured to retrieve the pipette tip from the second chamber and dispense at least a portion of the mixture onto the test strip using the pipette tip while the test strip is located at the second location;
an imaging system configured to capture an image of the test strip while the test strip is located at the third location;
a display configured to display instructions regarding using the test cartridge, the test strip, or the kiosk; and
a processor communicatively coupled to the vortex mixer, the conveyor belt, the robotic pipette module, the imaging system, and the display, wherein the processor is configured to execute instructions stored within a memory to:
   operate the vortex mixer;
   operate the conveyor belt;
   operate the robotic pipette module;
   operate the imaging system;
   receive the image of the test strip from the imaging system;
   analyze the image of the test strip to determine whether at least one of the one or more patient conditions is present; and
   operate the display.

EEE 40 is a method comprising:

receiving, by a kiosk, a test cartridge comprising:
a first chamber configured to store a patient sample and a buffer extraction solution; and
a second chamber configured to store a pipette tip;

receiving, by the kiosk, a test strip usable to indicate the presence of one or more patient conditions;

displaying, by a display of the kiosk, instructions regarding using the test cartridge, the test strip, or the kiosk; and

processing, by the kiosk, the test cartridge and the test strip, wherein processing the test cartridge and the test strip comprises:
mixing, by a vortex mixer, the patient sample with the buffer extraction solution to generate a mixture;
receiving, by a conveyor belt, the test strip at a first location within the kiosk;
transferring, by the conveyor belt, the test strip from the first location to a second location within the kiosk;
retrieving, by a robotic pipette module, the pipette tip from the second chamber;
dispensing, by the robotic pipette module, at least a portion of the mixture onto the test strip using the pipette tip while the test strip is located at the second location;
transferring, by the conveyor belt, the test strip from the second location to a third location within the kiosk;
capturing, using an imaging system, an image of the test strip while the test strip is located at the third location;
receiving, by a processor executing instructions stored within a memory, the image of the test strip from the imaging system; and
analyzing, by the processor executing the instructions stored within a memory, the image of the test strip to determine whether at least one of the one or more patient conditions is present.

1. A system comprising:
a test cartridge comprising:
a first chamber configured to store a patient sample and a buffer extraction solution; and
a second chamber configured to store a pipette tip;
a test strip usable to indicate the presence of one or more patient conditions; and
a kiosk configured to receive and process the test cartridge and the test strip, wherein the kiosk comprises:
a vortex mixer configured to mix the patient sample with the buffer extraction solution to generate a mixture;
a conveyor belt configured to:
receive the test strip at a first location within the kiosk;
transfer the test strip from the first location to a second location within the kiosk; and
transfer the test strip from the second location to a third location within the kiosk;
a robotic pipette module configured to retrieve the pipette tip from the second chamber and dispense at least a portion of the mixture onto the test strip using the pipette tip while the test strip is located at the second location;
an imaging system configured to capture an image of the test strip while the test strip is located at the third location;
a display configured to display instructions regarding using the test cartridge, the test strip, or the kiosk; and
a processor communicatively coupled to the vortex mixer, the conveyor belt, the robotic pipette module, the imaging system, and the display, wherein the processor is configured to execute instructions stored within a memory to:
operate the vortex mixer;
operate the conveyor belt;
operate the robotic pipette module;
operate the imaging system;
receive the image of the test strip from the imaging system;
analyze the image of the test strip to determine whether at least one of the one or more patient conditions is present; and
operate the display.

2. The system of claim 1, wherein the test cartridge comprises an ergonomic shell, wherein the ergonomic shell is made of moisture-impermeable material, and wherein the moisture-impermeable material comprises a seal layer and a desiccant.

3. The system of claim 2, wherein the robotic pipette module is configured to pierce the seal layer in order to access one or more components of the test cartridge.

4. The system of claim 2, wherein the test strip is configured to be stored within the test cartridge, and wherein the seal layer is configured to encapsulate the test strip.

5. The system of claim 1, wherein test cartridge comprises an external label, wherein the external label comprises a barcode indicative of a test cartridge number associated with the test cartridge, a test order number, and a test kit identification number, and wherein the test cartridge number is usable to link results of the image analysis performed by the processor to a patient associated with the patient sample.

6. The system of claim 1, wherein the test strip is configured to be stored within the test cartridge, and wherein:
the conveyor belt receiving the test strip at the first location comprises receiving the test cartridge at the first location;
the conveyor belt transferring the test strip from the first location to the second location comprises transferring the test cartridge from the first location to the second location; and
the conveyor belt transferring the test strip from the second location to the third location comprises transferring the test cartridge from the second location to the third location.

7. The system of claim 6, wherein the conveyor belt is further configured to, upon the imaging system capturing the image of the test strip while the test strip is located at the third location, transfer the test cartridge from the third location to the second location, and wherein the robotic pipette module is further configured to:
retrieve a wash buffer using the pipette tip; and
dispense the wash buffer into first chamber using the pipette tip.

8. The system of claim 7, wherein the test cartridge further comprises a third chamber configured to store the wash buffer, and wherein retrieving the wash buffer using the pipette tip comprises retrieving the wash buffer from the third chamber using the pipette tip.

9. The system of claim 1, wherein the first chamber comprises a vial configured to store the patient sample and the buffer extraction solution, and wherein the vial comprises a self-sealing cap.

10. The system of claim 1, wherein the conveyor belt comprises a plurality of slots, and wherein each slot of the plurality of slots is configured to hold a test strip or test cartridge.

11. The system of claim 1, wherein the robotic pipette module comprises:
one or more linear actuators configured to translate along one or more cartesian coordinate directions; and
one or more rotational actuators configured to rotate about one or more rotational axes.

12. The system of claim 1, wherein the imaging system comprises:
one or more light-emitting diodes (LEDs); and
one or more cameras.

13. The system of claim 1, wherein the kiosk further comprises a communication interface configured to communicate with one or more external computing devices over a network.

14. The system of claim 13, wherein the one or more external computing devices comprise a cloud-based storage unit, and wherein communicating with the one or more external computing devices comprises providing, to the cloud-based storage unit:
results of the image analysis performed by the processor;
a test cartridge number associated with the test cartridge;
a test order number; or
a self-testing kit identification number.

15. The system of claim 13, wherein the kiosk communicates with the one or more external computing devices over the network according to a protocol that complies with one or more privacy regulations.

16. The system of claim 13, wherein the one or more computing devices comprises a computing device of a patient associated with the patient sample or a computing device associated with a physician of the patient associated with the patient sample.

17. The system of claim 13, wherein the network comprises the public Internet.

18. The system of claim 1, wherein the robotic pipette module comprises a plunger configured to engage the vortex mixer to cause the vortex mixer to mix the patient sample with the buffer extraction solution to generate the mixture.

19. A kiosk configured to receive and process a test cartridge and a test strip, wherein the kiosk comprises:
a vortex mixer configured to mix a patient sample with a buffer extraction solution to generate a mixture, wherein the test cartridge comprises:
a first chamber configured to store the patient sample and the buffer extraction solution; and
a second chamber configured to store a pipette tip;
a conveyor belt configured to:
receive the test strip at a first location within the kiosk, wherein the test strip is usable to indicate the presence of one or more patient conditions;
transfer the test strip from the first location to a second location within the kiosk; and
transfer the test strip from the second location to a third location within the kiosk;
a robotic pipette module configured to retrieve the pipette tip from the second chamber and dispense at least a portion of the mixture onto the test strip using the pipette tip while the test strip is located at the second location;
an imaging system configured to capture an image of the test strip while the test strip is located at the third location;
a display configured to display instructions regarding using the test cartridge, the test strip, or the kiosk; and
a processor communicatively coupled to the vortex mixer, the conveyor belt, the robotic pipette module, the imaging system, and the display, wherein the processor is configured to execute instructions stored within a memory to:
operate the vortex mixer;
operate the conveyor belt;
operate the robotic pipette module;
operate the imaging system;
receive the image of the test strip from the imaging system;
analyze the image of the test strip to determine whether at least one of the one or more patient conditions is present; and
operate the display.

20. A method comprising:
receiving, by a kiosk, a test cartridge comprising:
a first chamber configured to store a patient sample and a buffer extraction solution; and
a second chamber configured to store a pipette tip;
receiving, by the kiosk, a test strip usable to indicate the presence of one or more patient conditions;
displaying, by a display of the kiosk, instructions regarding using the test cartridge, the test strip, or the kiosk; and
processing, by the kiosk, the test cartridge and the test strip, wherein processing the test cartridge and the test strip comprises:
mixing, by a vortex mixer, the patient sample with the buffer extraction solution to generate a mixture;
receiving, by a conveyor belt, the test strip at a first location within the kiosk;
transferring, by the conveyor belt, the test strip from the first location to a second location within the kiosk;
retrieving, by a robotic pipette module, the pipette tip from the second chamber;
dispensing, by the robotic pipette module, at least a portion of the mixture onto the test strip using the pipette tip while the test strip is located at the second location;
transferring, by the conveyor belt, the test strip from the second location to a third location within the kiosk;
capturing, using an imaging system, an image of the test strip while the test strip is located at the third location;
receiving, by a processor executing instructions stored within a memory, the image of the test strip from the imaging system; and
analyzing, by the processor executing the instructions stored within a memory, the image of the test strip to determine whether at least one of the one or more patient conditions is present.
